(19) United States
(12) Patent Application Publication
(10) Pub. No.: US 2021/0169939 A1
(43) Pub. Date: Jun. 10, 2021

(54) EXTRACELLULAR VESICLES AND THEIR USES

(71) Applicant: United Therapeutics Corporation, Silver Spring, MD (US)

(72) Inventors: Roger Ilagan, Durham, NC (US); Sarah Hogan, Raleigh, NC (US); John Cheadle, Durham, NC (US); Carolina Papu, Raleigh, NC (US)

(73) Assignee: United Therapeutics Corporation, Silver Spring, MD (US)

(21) Appl. No.: 17/109,775

(22) Filed: Dec. 2, 2020

Related U.S. Application Data
(60) Provisional application No. 62/943,555, filed on Dec. 4, 2019.
(60) Provisional application No. 63/003,521, filed on Apr. 1, 2020.

(51) Int. Cl. A61K 35/28; C12N 5/0775; A61P 11/00; A61K 9/00; A61K 31/519; A61P 9/12

(52) U.S. Cl. CPC A61K 35/28; C12N 5/0662; A61P 9/12; A61K 9/0029; A61K 31/519; A61P 11/00

(57) ABSTRACT

Provided are methods for isolating potent extracellular vesicle populations from mesenchymal stem cells. In particular, the present disclosure identified a protein profile specific for MSC-derived EV populations. Moreover, disclosed herein is the use of the isolated extracellular vesicles in treating a variety of diseases and conditions, including chronic or acute lung diseases such as pulmonary hypertension, ARDS and diseases and conditions characterized by vasculopathy, reduced angiogenesis, apoptosis, mitochondrial dysfunction, acute inflammation, fibrosis, or chronic inflammation.

### CROSS-REFERENCE TO RELATED APPLICATIONS

This application claims priority to U.S. Provisional Application Nos. 62/943,555, filed Dec. 4, 2019 and 63/003,521, filed Apr. 1, 2020.

### FIELD OF USE

The present application relates to extracellular vesicles, including exosomes, methods of isolating, engineering, or synthesizing potent extracellular vesicles, and the use of extracellular vesicles in treatment of acute and chronic lung diseases, including pulmonary hypertension, pulmonary arterial hypertension (PAH), and bronchopulmonary dysplasia, and conditions and diseases associated with inflammation, reduced angiogenesis, apoptosis, mitochondrial dysfunction, and vasculopathy. This application further relates to treating or preventing acute respiratory distress syndrome (ARDS) or acute lung injury (ALI) and treating or preventing fibrosis using extracellular vesicles, including exosomes.

### BACKGROUND

Mesenchymal stem cells (MSCs) are a heterogeneous fibroblast-like population of cells that can be isolated from many human tissues, including, but not limited to, bone marrow, adipose, skeletal muscle, heart, umbilical cord, and placenta. MSCs have attracted the attention of scientists and clinicians due to their differentiation potential and active participation in tissue repair and regeneration after migration to the site of tissue injury. When stimulated by appropriate signals, MSCs are capable of differentiating into a number of specialized cell types such as adipocytes, osteoblasts, chondrocytes, and, less frequently, endothelial cells and cardiomyocytes. MSCs are also amenable to allogeneic transplantation and are immunoprivileged, providing greater acceptance in vivo.

In addition, MSCs possess strong immunosuppressive and immunomodulatory properties that are mediated by both cell-cell contact and production of various signaling factors. Mesenchymal stem cells (MSCs) are able to migrate towards sites of inflammation/injury by sensing inflammatory cytokines. These cells are hypothesized to immunomodulate the inflammatory environment by releasing anti-inflammatory soluble factors as well as extracellular vesicles (EVs), including exosomes (paracrine effect). Moreover, the EVs themselves promote biological activities such as promoting angiogenesis, preventing apoptosis, reducing inflammation, and improving mitochondrial functions.

Bronchopulmonary Dysplasia (BPD) is a chronic lung disease of premature infants. It is characterized by prolonged lung inflammation, decrease in number of alveoli and thickened alveolar septae, abnormal vascular growth with “pruning” of distal blood vessels, and limited metabolic and antioxidant capacity. There are 14,000 new cases of BPD per year in the US. Importantly, a diagnosis of BPD often leads to other further conditions, including PH, emphysema, asthma, increase cardiovascular morbidity and post-neonatal mortality, increased neurodevelopmental impairment and cerebral palsy, emphysema as young adults. Currently, there is no standard therapy for BPD. Some BPD patients are treated with gentle ventilation and corticosteroids, but these treatments show no effects on neuro outcomes or death.

Pulmonary hypertension is a progressive and often fatal disease characterized by increased pressure in the pulmonary vasculature. Constriction of the pulmonary vasculature leads to increased stress on the right heart, which may develop into right heart failure. By definition under current standards, the mean pulmonary arterial pressure (mPAP) in a case of chronic pulmonary hypertension is >25 mmHg at rest or >30 mmHg during exertion (normal value<20 mmHg). Pulmonary arterial hypertension, untreated, leads to death on average within 2.8 to 5 years after diagnosis (Keily et al. (2013) BMJ 346:f2028). The pathophysiology of pulmonary arterial hypertension is characterized by vasoconstriction and remodeling of the pulmonary vessels. In chronic PAH there is neomuscularization of initially unmuscularized pulmonary vessels, and the vascular muscles of the already muscularized vessels increase in circumference. This resulting increase in pulmonary arterial pressures results in progressive stress on the right heart, which leads to a reduced output from the right heart and eventually ends in right heart failure (M. Humbert et al., J. Am. Coll. Cardiol. 2004, 43, 13S-24S).

PAH is a rare disorder, with a prevalence of 1-2 per million. The average age of the patients has been estimated to be 36 years, and only 10% of the patients were over 60 years of age. Distinctly more women than men are affected (G. E. D'Alonzo et al., Ann. Intern. Med. 1991, 115, 343-349).

Numerous mechanisms have been implicated in the pathogenesis of PAH. Importantly, a suppression of global metabolism has been described downstream of aberrant mitochondrial glucose oxidation in this disease. Diminished mitochondrial function could unify many apparently unrelated abnormalities in PAH, such as the involvement of multiple cell types, the cancer-like proliferation of pulmonary vascular cells, and resistance of these cells to apoptosis. Despite evidence supporting the role of mitochondrial dysfunction in PAH, therapeutic targeting of mitochondrial function has proven difficult.

Acute respiratory distress syndrome (ARDS) is an often-fatal condition characterized by fluid buildup in the alveoli of the lungs. The fluid buildup prevents proper oxygenation of the lungs and can result in death. ARDS can be caused, for example, by infection (viral or bacterial), sepsis, acid aspiration, or trauma. For example, cases of ARDS are frequently associated with sepsis and pneumonia. Recently, ARDS has been suggested as the primary fatal pathology associated with COVID-19 as a result of SARS-CoV-2 infection. Symptoms of ARDS include shortness of breath, rapid breathing, decreased blood pressure, confusion, and/or lethargy. ARDS can result in primary fibrosis, a condition currently with very few treatment options.

Treatment of ARDS is typically largely supportive and aims to address the lack of oxygenation resulting from fluid buildup. Thus, common treatments include supplemental oxygen and, when necessary, mechanical ventilation. Medical care providers may also address the underlying pathology, e.g., the infection or injury.

Pulmonary fibrosis is characterized by lung tissue becoming damaged or scarred, which prevents normal function of the lung tissue. The scarring can sometimes be traced back to a specific injury, but when the cause of pulmonary fibrosis cannot be determined, which is common, the condition is referred to as idiopathic pulmonary fibrosis. Symptoms include shortness of breath, fatigue, and a dry cough. The severity of symptoms varies greatly, and in some cases, especially progressive pulmonary fibrosis, the condition can be fatal.

Thus, a need exists to develop improved therapeutic compositions and methods for treating BPD and vasculopathies such as pulmonary hypertension, ARDS and pulmonary fibrosis, including ARDS and pulmonary fibrosis caused by viral (such as coronavirus) or bacterial infection. In particular there is a great need for developing EVs with improved potency to promote angiogenesis, prevent apoptosis, inflammation, and/or improve mitochondrial function in patients.

### SUMMARY OF THE INVENTION

The present disclosure is directed to an isolated extracellular vesicle (EV), wherein the isolated EV contains one more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132. In some embodiments, the EV contains one or more proteins selected from the group consisting of CD44, CD109, NT5E, MMP2 and HSPA8.

In some embodiments, the isolated EV is engineered to contain the one or more proteins.

In some embodiments, the isolated EV is obtained from a cell. In some embodiments, the cell is selected from an immortalized cell line or a primary cell. In some embodiments, the cell is a mesenchymal stem cell (MSC). In some embodiments, the cell is a non-MSC. In some embodiments, the non-MSC comprises a fibroblast cell, or a macrophage cell.

In some embodiments, the isolated EV has an increased amount of the one or more protein markers compared to the average amount in all EVs obtained from the MSC. In some embodiments, the isolated EV contains at least 20% increased amount of the one or more protein markers compared to the average amount in all EVs obtained from the MSC.

In some embodiments, the MSC is isolated from Wharton's jelly, umbilical cord blood, placenta, peripheral blood, bone marrow, bronchoalveolar lavage (BAL), or adipose tissue.

In some embodiments, the isolated EV is a synthetic exosome produced in vitro.

In some embodiments, the synthetic exosome is a synthetic liposome.

In some embodiments, the isolated EV further comprises one or more of Syntenin-1, Flotillin-1, CD105, and/or major histocompatibility complex class I.

In some embodiments, the isolated EV further comprises a member of the tetraspanin family.

In some embodiments, the member of the tetraspanin family comprises CD63, CD81, and CD9.

In another aspect, the present disclosure relates to a method of isolating an extracellular vesicle (EV) having increased potency comprising engineering the EV to express one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132. In some embodiments, the EV expresses one or more proteins selected from the group consisting of CD44, CD109, NT5E, MMP2 and HSPA8.

In some embodiments, the engineering comprises selecting EVs exhibiting an increased amount of the one or more proteins.

In some embodiments, the engineering comprises genetically engineering a cell producing EVs to contain the one or more proteins.

In some embodiments, the cell producing the EV comprises an immortalized cell line, a primary cell, a mesenchymal stem cell (MSC), a fibroblast, or a macrophage.

In some embodiments, the engineering comprises producing synthetic EVs in vitro that contain the one or more proteins.

In some embodiments, the isolated EVs have a mean diameter of about 100 nm.

In some embodiments, at least 70% of the isolated EVs have a size between 50 nm and 350 nm.

In some embodiments, the isolated EV further comprises Syntenin-1, Flotillin-1, CD105, and/or Major Histocompatibility Complex class I.

In some embodiments, the isolated EV further comprises a member of the tetraspanin family.

In some embodiments, the member of the tetraspanin family comprises CD63, CD81, and CD9.

In some embodiments, the increased potency of the EVs comprises increased pyruvate kinase activity.

In some embodiments, the increased potency of the EVs comprises increased ATPase activity.

In another aspect, the present disclosure relates to a method of treating a lung disease, comprising administering to a subject in need thereof isolated extracellular vesicles (EVs) obtained from mesenchymal stromal cells, wherein the isolated extracellular vesicles comprise extracellular vesicles having an increased amount of one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132. In some embodiments, the EVs expresses one or more proteins selected from the group consisting of CD44, CD109, NTSE, MMP2 and HSPA8.

In some embodiments, the lung disease comprises a chronic lung disease or an acute lung disease.

In some embodiments, the lung disease is bronchopulmonary dysplasia.

In another aspect, the present disclosure relates to a method of treating a disease or condition associated with reduced angiogenesis, acute inflammation, chronic inflammation, apoptosis, mitochondrial dysfunction, or vasculopathy, comprising administering to a subject in need thereof isolated extracellular vesicles obtained from mesenchymal stromal cells, wherein the isolated extracellular vesicles comprise extracellular vesicles having increased expression of one or more of one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132.

In some embodiments, the EVs comprise one or more proteins selected from the group consisting of CD44, CD109, NTSE, and HSPA8.

In some embodiments, the isolated extracellular vesicles normalize glucose oxidation in lung tissue of the subject.

In some embodiments, the disease or condition associated with mitochondrial dysfunction is associated with decreased mitochondrial glucose oxidation in the subject.

In some embodiments, the disease or condition associated with mitochondrial dysfunction is selected from the group consisting of Friedreich's ataxia, Leber's Hereditary Optic Neuropathy, Kearns-Sayre Syndrome, Mitochondrial Encephalomyopathy with Lactic Acidosis and Stroke-Like Episodes, Leigh syndrome, obesity, atherosclerosis, amyotrophic lateral sclerosis, Parkinson's Disease, cancer, heart failure, myocardial infarction (MI), Alzheimer's Disease, Huntington's Disease, schizophrenia, bipolar disorder, fragile X syndrome, and chronic fatigue syndrome.

In another aspect, the present disclosure relates to a composition comprising extracellular vesicles (EVs) isolated from bone marrow mesenchymal stem cells (MSCs), wherein the EVs: (i) are substantially free from organelles or organelle fragments within the EVs; (ii) comprise lipids, proteins, nucleic acids, and cellular metabolites; (iii) have a weighted mean diameter of between 200-300 nm; and (iv) express one or more proteins selected from KRT19, TUBB, TUBB2A, TUBB2B, TUBB2C, TUBB3, TUBB4B, TUBB6, CFL1 (HEL-S-15), and VIM. In some embodiments, the EVs further express one or more proteins selected from EEF1A1, EEF1A1P5, PTI-1, EEF1A1L14, and EEFA2. In some embodiments, the EVs further express one or more proteins selected from ENPP1 and NT5E. In some embodiments, the EVs further express HSPA8. In some embodiments, the EVs further express CD44. In some embodiments, the EVs further express MMP2. In some embodiments, the EVs further express CD109.

In another aspect, the present disclosure relates to a composition comprising extracellular vesicles (EVs) isolated from bone marrow mesenchymal stem cells (MSCs), wherein the EVs: (i) are substantially free from organelles or organelle fragments within the EVs; (ii) comprise lipids, proteins, nucleic acids, and cellular metabolites; (iii) have a weighted mean diameter of between 200-300 nm; and (iv) express one or more proteins selected from KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, SPA8 (HEL-S-72p), RAB10, CD44, MMP2, CD109, and DKFZp686P132. In some embodiments, the EVs express one or more proteins selected from CD44, CD109, NT5E, MMP2 and HSPA8.

In one aspect, the present disclosure relates to a method of treating or preventing acute respiratory distress syndrome (ARDS) comprising administering to a subject in need thereof an effective dose of an isolated extracellular vesicle (EV) of any of the foregoing embodiments. Preferably, the isolated EV expresses one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132.

In some embodiments, the method treats ARDS resulting from an infection, sepsis, acid aspiration, or trauma. In some embodiments, the infection is a viral infection or a bacterial infection. In some embodiments, the infection is caused by a coronavirus. In some embodiments, the coronavirus comprises human coronavirus 229E, human coronavirus OC43, SARS-CoV, HCoV NL63, HKU1, MERS-CoV, or SARS-CoV-2. In some embodiments, the pulmonary fibrosis is the result of a SARS-CoV-2 infection.

In some embodiments, the method treats ARDS resulting from COVID-19.

In some embodiments, the method prevents or reduces severity ARDS.

In some embodiments, the subject is at risk of developing ALI or ARDS.

In some embodiments, the isolated EV are administered parenterally.

In some embodiments, the effective dose of the isolated EV is from about 20 to about 500 pmol phospholipid of EVs per kg of subject being treated.

In some embodiments, the method further comprises administering a therapeutic agent comprising one or more of a phosphodiesterase type-5 (PDE5) inhibitor, a prostacyclin agonist, or an endothelin receptor antagonist.

In some embodiments, the PDE5 inhibitor comprises sildenafil, vardenafil, zapravist, udenafil, dasantafil, avanafil, mirodenafil, or lodenafil.

In some embodiments, the PDE5 inhibitor is sildenafil.

In some embodiments, the prostacyclin agonist comprises epoprostenol sodium, treprostinil, beraprost, ilprost, and a PGI2 receptor agonist.

In some embodiments, the isolated EV and the phosphodiesterase type-5 (PDE5) inhibitor are administered in separate compositions, simultaneously or sequentially.

In some embodiments, the isolated EV and the phosphodiesterase type-5 (PDE5) inhibitor are administered in the same composition.

In some embodiments, the isolated EV are administered in one or more doses.

In some embodiments, the isolated EV is administered at an interval of 12 hours, 24 hours, 48 hours, 72 hours, 4 days, 5, days, 6 days, or once per week.

In some embodiments, the isolated EV is administered in 2 doses, 3 doses, 4 doses, 5 doses, 6 doses, 7 doses, 8 doses, 9 doses, 12 doses, 15 doses, or 18 doses.

In some embodiments, wherein the isolated EV and the therapeutic agent are administered in the same composition. In some embodiments, the isolated EV and the PDE5 inhibitor are administered in one or more doses. In some embodiments, the isolated EV and the prostacyclin agonist are administered in one or more doses. In some embodiments, the isolated EV and the endothelin receptor agonist are administered in one or more doses. The doses can be administered concurrently or separated in time.

In some embodiments, the isolated EV and therapeutic agent are administered at an interval of 6 hours, 12 hours, 24 hours, 48 hours, 72 hours, 4 days, 5, days, 6 days, or once per week.

In some embodiments, the isolated EV and the PDE5 inhibitor are administered at an interval of 6 hours, 12 hours, 24 hours, 48 hours, 72 hours, 4 days, 5, days, 6 days, or once per week.

In some embodiments, the isolated EV and the prostacyclin agonist are administered at an interval of 6 hours, 12 hours, 24 hours, 48 hours, 72 hours, 4 days, 5, days, 6 days, or once per week.

In some embodiments, the isolated EV and the endothelin receptor are administered at an interval of 6 hours, 12 hours, 24 hours, 48 hours, 72 hours, 4 days, 5, days, 6 days, or once per week.

In some embodiments, the isolated EV is administered in 2 doses, 3 doses, 4 doses, 5 doses, 6 doses, 7 doses, 8 doses, 9 doses, 12 doses, 15 doses, or 18 doses, and wherein the PDE5 inhibitor is administered in 16 doses, 19 doses, 21 doses, 24 doses, 27 doses, 30 doses, 33 doses, 36 doses, 39 doses, 42 doses, 45 doses, 48 doses, 51 doses, 54 doses, 57 doses, 60 doses, 63 doses, or 66 doses.

In some embodiments, the isolated EV is administered once daily for 2 days, for 3 days, for 4 days, for 5 days for 6 days, or for a week.

In some embodiments, the method decreases systolic pulmonary arterial pressure (SPAP) in the subject.

In some embodiments, the method increases alveolar surface area and/or reduces alveolar damage of the lung in the subject.

In some embodiments, the method increases a concentration of blood oxygen in the subject.

In some embodiments, the method reduces inflammation in the lung in the subject.

In some embodiments, the method reduces deposition of extracellular matrix in the bronchoalveolar lavage fluid.

In some embodiments, the method improves Fulton's index or pulmonary vascular remodeling.

In some embodiments, the subject is a human, a non-human primate, a dog, a cat, a cow, a sheep, a horse, a rabbit, a mouse, or a rat.

In another aspect, the present disclosure relates to a method of treating or preventing pulmonary fibrosis comprising administering to a subject in need thereof an effective dose of isolated extracellular vesicles (EV), wherein the isolated EV contains one more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132.

In some embodiments, the pulmonary fibrosis is idiopathic pulmonary fibrosis.

In some embodiments, the pulmonary fibrosis is the result of an infection. In some embodiments, the infection is caused by a coronavirus. In some embodiments, the coronavirus comprises human coronavirus 229E, human coronavirus OC43, SARS-CoV, HCoV NL63, HKU1, MERS-CoV, or SARS-CoV-2. In some embodiments, the pulmonary fibrosis is the result of a SARS-CoV-2 infection.

In some embodiments, the method comprises administering EV to a patient at risk of developing pulmonary fibrosis.

In another aspect, the present disclosure relates to a method of treating a respiratory disease or disorder, comprising administering to a subject in need thereof an effective dose of an isolated extracellular vesicle (EV) and a phosphodiesterase type-5 (PDE5) inhibitor, wherein the isolated EV contains one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132.

In some embodiments, the respiratory disease or disorder comprises acute respiratory distress syndrome (ADRS), acute lung disease, acute lung injury (ALI), asthma, chronic obstructive pulmonary disease, cystic fibrosis, pneumonitis, pulmonary fibrosis, acute lung injury, bronchitis, emphysema, bronchiolitis obliterans, or bronchopulmonary dysplasia (BPD).

In some embodiments, the method treats or prevents respiratory disease or disorder resulting from COVID-19.

In some embodiments, the pulmonary fibrosis is idiopathic pulmonary fibrosis.

In some embodiments, the respiratory disease or disorder is the result of an infection.

In some embodiments, the respiratory disease or disorder is the result of a SARS-CoV-2 infection.

In some embodiments, the method comprises administering EV to a patient at risk of developing respiratory disease or disorder.

### BRIEF DESCRIPTION OF THE DRAWINGS

The provided drawings exemplify, but do not limit, the disclosed subject matter.

FIG. 1 shows isolation of the UNEX-42 development grade extracellular vesicles (EVs) from cell culture supernatant of bone marrow mesenchymal stem cells (MSC). FIG. 1A shows the chromatographic profiles of the UNEX-42 EVs and reference polystyrene beads with diameters of 100 nm (Phosphorex™, 4002) and 200 nm (Phosphorex™, 2202) generated during the size exclusion chromatography (SEC) purification step. FIG. 1B shows the chromatographic profiles of different EV populations isolated by size exclusion chromatography.

FIG. 2 shows a representative size distribution from the UNEX18-015 batch of UNEX-42 EVs generated by nanoparticle tracking analysis (NTA).

FIG. 3 shows a graph demonstrating that the phospholipid content of UNEX-42 EV batches is proportional to particle count.

FIG. 4 shows proteomic profiling of the UNEX-42 EV development-grade batches generated by UPLC-MS/MS mass spectrometry. FIG. 4A shows heat map analysis of the mass spectrometry results. FIG. 4B shows a comparison of 142 sequences found to be common among all UNEX-42 EV batches with proteins present in fibroblast-derived EVs to determine UNEX-42 EV specific proteins.

FIG. 5 shows a histogram depicting frequencies of tetraspanin-positive particles in UNEX-42 EV batches.

FIG. 6 shows an illustrative structural schematic of UNEX-42 EVs.

FIG. 7 shows that UNEX-42 EVs prevents Cytochrome C release and cellular death after hyperoxia exposure. PBS is abbreviation for phosphate buffered saline. *p<0.05 represents hyperoxia compared to normoxia treatments. #p<0.05 represents hyperoxia+UNEX-42 treatment compared to hyperoxia.

FIG. 8 shows that UNEX-42 EVs promote microvascular network formation in vitro. PBS is abbreviation for phosphate buffered saline. *p<0.05 represents normoxia compared to normoxia and UNEX-42 treatments.

FIG. 9 shows that UNEX-42 EVs prevent HPEAC network degradation following hyperoxia exposure. *p<0.05 represents hyperoxia compared to normoxia treatment. #p<0.05 represents hyperoxia and UNEX-42 treatment compared to hyperoxia.

FIG. 10 shows that UNEX-42 EVs preserve MMP-2 secretion and activity following Hyperoxia Exposure. MMP2 is abbreviation for matrix metalloproteinase 2. *p<0.05 represents hyperoxia compared to normoxia treatments. #p<0.05 represents hyperoxia+UNEX-42 treatment compared to hyperoxia.

FIG. 11 shows that UNEX-42 EVs increase oxygen consumption and glucose uptake and decrease lactate accumulation. FCCP is abbreviation for carbonyl cyanide p-triflouromethoxyphenylhydrazone; Gluc is abbreviation for glucose; H is the ratio of normoxia/hypoxia; H+E is the ratio of hypoxia+UNEX-42/hypoxia; Lac is abbreviation for lactate; OCR is abbreviation for oxygen consumption rate; Pyr is abbreviation for pyruvate; Rot-AA is abbreviation for rotenone/antimycin A. a indicates Hypoxia compared to normoxia treatment, p<0.05. b indicates Hypoxia+UNEX-42 treatment compared to hypoxia, p<0.05.

FIG. 12 shows that UNEX-42 EVs suppress Hyperoxia-Induced Secretion of Tumor Necrosis Factor Alpha. PBS is abbreviation for phosphate buffered saline; TNFa is abbreviation for tumor necrosis factor alpha. *p<0.05 represents hyperoxia compared to normoxia treatments. #p<0.05 represents hyperoxia+UNEX-42 treatment compared to hyperoxia.

FIG. 13 shows that UNEX-42 EVs inhibit LPS-induced Tumor Necrosis Factor Alpha (TNFα). FIG. 13A shows a graph depicting TNFα inhibition as a function of UNEX-42 concentration. FIG. 13B is a histogram showing TNFα secretion upon LPS treatment. LPS is abbreviation for lipopolysaccharide; TNFα is abbreviation for tumor necrosis factor alpha. *p<0.05 represents hyperoxia compared to normoxia treatment. #p<0.05 represents hyperoxia+UNEX-42 treatment.

FIG. 14 shows graphs depicting total cell count in BAL after hyperoxia exposure. UNEX-42 trends to decrease total cell count in the BAL after hyperoxia. BAL is abbreviation for bronchoalveolar lavage fluid. *p<0.05 represents hyperoxia compared to normoxia treatment.

FIG. 15 shows that UNEX-42 EVs improve Fulton Index after 10 days of hyperoxia exposure. *p<0.05 represents hyperoxia compared to normoxia treatment #p<0.05 represents hyperoxia+UNEX-42 treatment compared to hyperoxia.

FIG. 16 shows that UNEX-42 EVs improve lung histology after hyperoxia exposure.

FIG. 17 shows that UNEX-42 EVs improve MLI after Hyperoxia Exposure. MLI is abbreviation for mean linear intercept. *p<0.05 represents hyperoxia compared to normoxia treatment. #p<0.05 represents hyperoxia+UNEX-42 treatment compared to hyperoxia.

FIG. 18 shows that UNEX-42 EVs improve tidal volume after hyperoxia exposure. TVb is abbreviation for tidal volume. *p<0.05 represents hyperoxia compared to normoxia treatment. #p<0.05 represents hyperoxia+UNEX-42 treatment compared to hyperoxia.

FIG. 19 shows the effect of UNEX-42 EVs and sildenafil on systolic pulmonary artery pressure (SPAP). SPAP was measured in a semaxinib/Hypoxia rat model. G1 indicates the DMSO disease control. G2-G7 indicates groups of rats exposed to semaxinib and Hypoxia. G3 indicates Sildenafil treatment. G4-G6 indicate groups treated with the indicated dosage of UNEX-42 EVs. G7 indicates combination treatment of UNEX-42 EVs and sildenafil.

FIG. 20 shows the effect of UNEX-42 EVs and sildenafil on systolic pulmonary artery pressure (SPAP). SPAP was measured in a semaxinib/Hypoxia rat model. G1 indicates the DMSO disease control. G2-G7 indicates groups of rats exposed to semaxinib and Hypoxia. G3 indicates Sildenafil treatment. G4-G6 indicate groups treated with a combination of UNEX-42 EVs and sildenafil with varying doses of UNEX-42 EVs as indicated. G7 indicates a group treated with only UNEX42 EVs at the indicated dosage.

FIG. 21 shows that UNEX-42 EVs improve MLI after Hyperoxia Exposure (A) and increased blood oxygen levels after hyperoxia (B). MLI is an abbreviation for mean linear intercept.

FIG. 22 shows that UNEX-42 EVs reduce number of immune cells infiltrating the bronchoalveolar lavage (BAL) in a bleomycin (Bleo) model for idiopathic pulmonary fibrosis (IPF).

FIG. 23 shows that UNEX-42 EVs reduce total number of cells (A) and the number of macrophages, lymphocytes, and neutrophils (B) infiltrating the bronchoalveolar lavage fluid (BALF) in a silica model for pulmonary fibrosis.

FIG. 24 shows that UNEX-42 EVs suppress hyperoxia-induced secretion of TNFa (A), secretion of hyperoxia-induced IL6 (B), and secretion of hyperoxia-induced IL3 (C).

FIG. 25 shows that UNEX-42 EVs attenuate LPS-Induced Chemokine (C-X-C Motif) Ligand 1 (GRO).

FIG. 26 shows that UNEX-42 EVs attenuate LPS-Induced Chemokine (C-C Motif) Ligand 21 (6CKine).

FIG. 27 shows that UNEX-42 EVs attenuate LPS-Induced Granulocyte Chemotactic Protein 2 (GCP2).

FIG. 28 shows that UNEX-42 EVs attenuate LPS-Induced Chemokine (C-X-C Motif) Ligand 16 (CXCL16).

FIG. 29 shows that UNEX-42 EVs inhibit LPS-Induced TNFa Secretion in Mouse Monocytes.

FIG. 30 shows that UNEX-42 EVs inhibit LPS-Induced TNFa and Chemokine (C-X-C Motif) Ligand 1 (GRO) secretion in rat peripheral blood mononuclear cells (PBMCs).

FIG. 31 shows that UNEX-42 EVs attenuated LPS-induced mRNA expression of interleukin 1 beta (IL1β) (A) and interleukin 12 beta (IL12β) (B) in human THP1 monocytes. FIG. 31 (C) shows that UNEX-42 EVs attenuated secretion of the pro-inflammatory cytokines macrophage inflammatory protein 1 alpha (MIP1α) and beta (MIP1β).

FIG. 32 shows that UNEX-42 (19-017) EVs improve MLI after Hyperoxia Exposure. MLI is abbreviation for mean linear intercept. UNEX-42 EVs were administered at a dose of 125 nM phospholipid.

FIG. 33 shows that UNEX-42 EVs treatment induces expression of anti-inflammatory CD206 mRNA (A) and IL10 mRNA (B).

FIG. 34 shows that UNEX-42 EVs treatment improves total cell count (A) and the number of macrophages, lymphocytes, and neutrophils (B) in bronchoalveolar lavage (BAL) in a bleomycin (Bleo) model for idiopathic pulmonary fibrosis (IPF).

FIG. 35 shows that UNEX-42 EVs treatment reduced soluble collagen in bronchoalveolar lavage (BAL) in a bleomycin (Bleo) model for idiopathic pulmonary fibrosis (IPF).

FIG. 36 shows that UNEX-42 EVs treatment improves Fulton's index after 8 days of hyperoxia exposure.

### DETAILED DESCRIPTION

Extracellular vesicles (EVs) from mesenchymal stem cells (MSCs) can have a number of potentially beneficial physiological effects. Specifically, the EVs can enhance glucose oxidation and normalize mitochondrial function. In the context of pulmonary physiology, EVs can decrease systolic pulmonary artery pressure (SPAP), increase alveolar surface area, increase blood oxygen, reduce deposition of extracellular matrix protein, improve Fulton's index, and reduce inflammation in the lung of a subject. Thus, these extracellular vesicles can confer therapeutic benefit in pulmonary arterial hypertension (PAH), respiratory distress diseases or conditions such as ARDS and pulmonary fibrosis, and diseases or conditions associated with mitochondrial dysfunction. The present disclosure provides EVs, methods of obtaining EVs, and methods of treating or preventing respiratory distress diseases or conditions such as ARDS and pulmonary fibrosis, including ARDS and pulmonary fibrosis associated with COVID-19 or SARS-CoV or a related coronavirus infection, and a variety of other diseases and conditions using these EVs. In one embodiment, the present disclosure provides methods for treating or preventing idiopathic pulmonary fibrosis.

In particular, the inventors engineered and isolated EVs from MSCs and performed proteomics analysis of the EVs derived from MSCs to elucidate the structure of the EVs and identify components affecting potency. Identification of the proteins differentially contained in EVs derived from MSC enables production of biologically-engineered EVs or synthetic EVs that can mimic these cell derived EVs.

The proteins differentially contained in the EVs derived from MSC can comprise one or more cytoskeletal proteins, one or more gene transcription/translation related proteins, one or more nucleases or nucleotidases, one or more heat shock proteins, one or more vesicle trafficking related proteins, one or more extracellular matrix (ECM) related proteins, one or more proteolysis related proteins, and one or more cell signaling proteins. See Table 3 in Example 1.4. In particular, the one or more cytoskeletal proteins identified to be differentially contained in MSC-derived EVs and exosomes comprise Keratin type I cytoskeletal 19 (KRT19), tubulin beta chain (TUBB), tubulin beta chain 2A (TUBB2A), tubulin beta chain (TUBB2B), tubulin beta chain 2C (TUBB2C), tubulin beta chain 3 (TUBB3), tubulin beta chain 4B (TUBB4B), tubulin beta chain 6 (TUBB6), cofilin 1 (CFL1 or HEL-S-15), and vimentin (VIM). The one or more gene transcription related proteins identified to be differentially contained in MSC-derived EVs and exosomes comprise eukaryotic elongation factor 1 (EEF1A1), eukaryotic elongation factor 1 alpha pseudogene 5 (EEF1A1P5), prostate tumor inducing 1 (PTI-1), eukaryotic elongation factor 1 alpha 1-like 14 (EEF1A1L14), and eukaryotic elongation factor alpha 2 (EEFA2). The one or more nuclease identified to be differentially contained in MSC-derived EVs and exosomes comprise ectonucleotide pyrophospatase/phosphodiesterase 1 (ENPP1), and 5′-nucleotidase ecto (NTSE). Other proteins identified to be identified to be differentially contained in MSC-derived EVs and exosomes comprise heat shock protein A8 (HSPA8 or HEL-S-72p or Hsc70), RAB10 (a small GTPase protein involved in vesicular trafficking), CD44 (a cell surface adhesion receptor that interacts with extracellular matrix components such as hyaluronan), matrix metalloproteinase 2 (MMP2), CD109 (inhibitor of TGF-beta receptor signaling), and unknown protein DKFZp686P132.

Accordingly, in some embodiments, the present disclosure provides an isolated EV, wherein the isolated EV contains one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132. In some preferred embodiments, the EV contains one or more proteins selected from the group consisting of CD44, CD109, NT5E, and HSPA8.

In another aspect, the present disclosure relates to a composition comprising extracellular vesicles (EVs) isolated from bone marrow mesenchymal stem cells (MSCs), wherein the EVs: (i) are substantially free from organelles or organelle fragments within the EVs; (ii) comprise lipids, proteins, nucleic acids, and cellular metabolites; (iii) have a weighted mean diameter of between 200-300 nm; and (iv) contain one or more proteins selected from KRT19, TUBB, TUBB2A, TUBB2B, TUBB2C, TUBB3, TUBB4B, TUBB6, CFL1 (HEL-S-15), and VIM. In some embodiments, the EVs further contain one or more proteins selected from EEF1A1, EEF1A1P5, PTI-1, EEF1A1L14, and EEFA2. In some embodiments, the EVs further contain one or more proteins selected from ENPP1 and NT5E. In some embodiments, the EVs further contain HSPA8. In some embodiments, the EVs further contain CD44. In some embodiments, the EVs further contain MMP2. In some embodiments, the EVs further contain CD109.

In another aspect, the present disclosure relates to a composition comprising extracellular vesicles (EVs) isolated from bone marrow mesenchymal stem cells (MSCs), wherein the EVs: (i) are substantially free from organelles or organelle fragments within the EVs; (ii) comprise lipids, proteins, nucleic acids, and cellular metabolites; (iii) have a weighted mean diameter of between 200-300 nm; and (iv) contain one or more proteins selected from KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, SPA8 (HEL-S-72p), RAB10, CD44, MMP2, CD109, and DKFZp686P132. In some embodiments, the EVs contain one or more proteins selected from CD44, CD109, NT5E, MMP2 and HSPA8.

It is also contemplated herein that the present disclosure can be applied in treating any respiratory disease or disorder. For example, the respiratory disease or disorder may include acute respiratory distress syndrome (ADRS), acute lung disease, asthma, chronic obstructive pulmonary disease, cystic fibrosis, pneumonitis, pulmonary fibrosis, acute lung injury, bronchitis, emphysema, bronchiolitis obliterans, or bronchopulmonary dysplasia (BPD). In some embodiments, the method treats or prevents respiratory disease or disorder resulting from coronavirus infection or COVID-19. Because of the physiological effects from EVs, the EVs can be used to treat or prevent lung conditions characterized by inflammatory processes or decreased oxygenation of the blood caused by pulmonary dysfunction.

### A. Definitions

Unless otherwise specified, “a” or “an” means “one or more.”

Unless specifically defined otherwise, all technical and scientific terms used herein shall be taken to have the same meaning as commonly understood by one of ordinary skill in the art.

Unless otherwise indicated, the molecular biology, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

The process of obtaining EVs containing these proteins of interest is generally referred to herein as “engineering” the EVs to contain one more desired proteins, including proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132. As used herein, the term “engineering” is meant to broadly refer to any possible means of obtaining EVs that contain the desired proteins. The term “engineering” includes any form of manipulation, selection, isolation, culturing, or purification of either the EV directly or the donor cell the EV is derived from to generate EVs containing increased levels of one or more of the proteins herein identified to be differentially contained in EVs derived from MSC. Illustrative embodiments of engineering EVs to contain the desired proteins are further described below.

As used herein, ARDS means acute respiratory distress syndrome. ARDS can be caused, for example, by infection (viral or bacterial), sepsis, acid aspiration, or trauma. The ARDS can have an unknown cause. The ARDS can be associated with COVID-19 or SARS-CoV infection.

As used herein, pulmonary fibrosis refers to the condition characterized by scarring or damage to lung tissue. Pulmonary fibrosis includes fibrosis with any cause or an unknown cause (idiopathic pulmonary fibrosis). The pulmonary fibrosis can be associated with COVID-19 infection or SARS-CoV infection.

The ARDS, pulmonary fibrosis or related respiratory diseases or disorders can also be associated with infections by a coronavirus. In some embodiments, the coronavirus comprises human coronavirus 229E, human coronavirus OC43, SARS-CoV, HCoV NL63, HKU1, MERS-CoV, or SARS-CoV-2. In some embodiments, the ARDS, pulmonary fibrosis or related respiratory diseases or disorders may be associated with any infectious diseases or disorders.

As used herein, the term “subject” (also referred to herein as a “patient”) includes warm-blooded animals, preferably mammals, including humans. In a preferred embodiment, the subject is a primate. In an even more preferred embodiment, the subject is a human.

As used herein the terms “treating”, “treat,” or “treatment” include reducing, mitigating, or eliminating at least one symptom of a disease or condition.

As used herein the terms “preventing”, “prevent” or “prevention” include stopping or hindering the appearance or existence of at least one symptom of a disease or condition, such as vasculopathy. Alternatively, the terms “preventing”, “prevent” or “prevention” may include stopping or hindering the appearance or existence of at least one symptom of a disease or condition, such as dysfunctional angiogenesis, apoptosis, inflammation, mitochondrial dysfunction.

As used here, the term “expression” means RNA expression and/or protein expression level of one or more genes. In other words, the term “expression” can refer to either RNA expression or protein expression or a combination of the two. As used herein, the term contain or containing may include protein and/or RNA expression.

As used here, the term “hypoxia” refers to a condition with an oxygen (O₂) concentration below atmospheric O₂ concentration, 21%. In some embodiments, hypoxia refers to a condition with O₂ concentration that is between 0% and 10%, between 0% and 5% O₂, between 5% and 10%, or between 5% and 15%. In one embodiment, hypoxia refers to a concentration of oxygen of about 10% O₂.

As used here, the term “normoxia” refers to a condition with a normal atmospheric concentration of oxygen, around 20% to 21% O₂.

As used here, the terms “isolating” or “isolated,” when used in the context of an extracellular vesicle isolated from a cell culture or media, refers to an extracellular vesicle that, by the hand of man, exists apart from its native environment.

As used here, the term “extracellular vesicles”, abbreviated as EVs, encompass exosomes. The terms “extracellular vesicles” and “EVs,” as used herein, may in some embodiments refer to a membranous particle having a diameter (or largest dimension where the particles is not spheroid) of between about 10 nm to about 5000 nm, more typically between 30 nm and 1000 nm, and most typically between about 50 nm and 750 nm. Most commonly, EVs will have a size (average diameter) that is up to 5% of the size of the donor cell. Therefore, especially contemplated EVs include those that are shed from a cell.

As used here, the term “population of extracellular vesicles” refers to a population of extracellular vesicles having a distinct characteristic or set of characteristics. The terms “population of extracellular vesicles” and “extracellular vesicles” can be used interchangeably to refer to a population of extracellular vesicles having a distinct characteristic or set of characteristics.

As used here, the term “mesenchymal stromal cell” includes mesenchymal stem cells. Mesenchymal stem cells are cells found in bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum. Mesenchymal stem cells are capable of differentiating into a large number of cell types including, but not limited to, adipose, osseous, cartilaginous, elastic, muscular, and fibrous connective tissues. The specific lineage-commitment and differentiation pathway entered into by mesenchymal stem cells depends upon various influences, including mechanical influences and/or endogenous bioactive factors, such as growth factors, cytokines, and/or local micro environmental conditions established by host tissues. Mesenchymal stem cells are thus non-hematopoietic progenitor cells that divide to yield daughter cells that are either stem cells or are precursor cells which in time will irreversibly differentiate to yield a phenotypic cell.

Some embodiments of the disclosure relate broadly to mesenchymal stromal cell extracellular vesicles, which are interchangeably referred to as mesenchymal stromal cell extracellular vesicles, or MSC extracellular vesicles, or extracellular vesicles.

It is also contemplated herein that the EV encompasses synthetic exosomes. As used herein, the term “synthetic exosome” refers to an exosome that is produced in vitro and not by cells. For example, a synthetic exosome may be a liposome formed by the generation of enclosed lipid bilayers or aggregates. Liposomes may be characterized as having vesicular structures with a bilayer membrane, generally comprising a phospholipid, and an inner medium that generally comprises an aqueous composition.

### B. Extracellular Vesicles

i. Engineering EVs to Contain MSC-Specific Proteins

The present disclosure provides methods of engineering MSC derived EVs having increased potency for treating PAH, PH, BPD or disorders associated with mitochondrial dysfunction, reduced angiogenesis, apoptosis, or inflammation. In some embodiments, the EVs may be derived from any cell type. In some embodiments, the EVs may be derived from immortalized cells or cells established as an immortal cell line. In some embodiments, the EVs may be derived from primary cells. As used herein, the term “primary cells” means cells directly obtained from a subject or a donor, and the primary cells have not been immortalized and/or established as an immortal cell line. In some preferred embodiments, the EVs are derived from a mesenchymal stem cell (MSC).

In some embodiments, present disclosure provides a method of isolating an extracellular vesicle (EV) having increased potency comprising engineering the EV to contain one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132, or more preferably, the EV may be engineered to contain one or more proteins selected from the group consisting of CD44, CD109, NT5E, MMP2 and HSPA8.

In some embodiments, the donor cell is a MSC and the EVs are isolated by using the differentially contained proteins as markers for selection. For example, the EVs may be selected from a MSC culture by using flow cytometry or panning or any other well-known immunoselection method to isolate EVs containing increased levels of one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132 compared to the average of all EVs derived from MSC. In some preferred embodiments, the EVs may be engineered to contain increased levels of one or more proteins selected from the group consisting of CD44, CD109, NT5E, MMP2 and HSPA8 compared to the average level of all EVs derived from the MSC.

In another aspect, the donor cell used to derive the EVs may not be a MSC and may not contain any of the proteins of interest. Rather, the non-MSC donor cell may be engineered genetically to contain the one or more of the proteins of interest by using standard molecular biology techniques as referenced elsewhere herein. Accordingly, in some embodiments, the EVs may be derived from a non-MSC cell genetically engineered to contain one or more proteins selected from KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132. In a preferred embodiment, the EVs may be derived from a non-MSC cell genetically engineered to contain one or more proteins selected from the group consisting of CD44, CD109, NTSE, MMP2 and HSPA8. The cell used as donor for deriving the EVs of the present disclosure may be any type of cell. In some embodiments, the non-MSC cell may be a fibroblast or a macrophage.

In another aspect, the EV may be directly engineered to contain the protein of interest. Various methods of directly engineering exosomes to contain a protein of interest are well known in the art. For example, the EVs may be incubated with isolated or purified proteins of interest. The EVs may be passively incubated together with the isolated or purified proteins of interest. To improve the efficiency of loading the EVs with the protein of interest, the mixture of EVs and an isolated protein of interest may be sonicated to compromise the membrane integrity and then let the EVs restore with the protein of interest embedded in the membrane. The EVs may also be engineered to contain a protein of interest by an extrusion protocol, wherein the EVs are mixed with the protein of interest and subsequently loaded into a syringe-based lipid extruder to vigorously mix the exosome and protein of interest.

In some embodiments, the EVs are engineered to contain the protein of interest by mixing the EVs with the protein of interest and subsequently and repeatedly freezing and thawing the mixture. The EVs may also be engineered to contain the protein of interest by electroporation or by incubation with permeabilizers that are well known in the art.

In some embodiments, the EVs are engineered to contain the protein of interest by using click chemistry approaches. For example, copper-catalyzed azide alkyne cycloaddition may be used to conjugate a protein of interest directly to the EVs.

By using the methods of engineering the EVs to contain a protein of interest as described herein, a synthetic EV or exosome such as a liposome may also be engineered to contain one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132, or in a preferred embodiment to contain one or more proteins selected from the group consisting of CD44, CD109, NTSE, MMP2 and HSPA8.

EVs can be further assessed for containing the protein markers Syntenin-1, Flotillin-1, CD105, Major histocompatibility complex class I, and members of the tetraspanin family. Accordingly, the isolated EVs further comprise Syntenin-1, Flotillin-1, CD105, and/or Major Histocompatibility complex class I. In some further embodiments, wherein the isolated EV further comprises a member of the tetraspanin family. In some embodiments, the member of the tetraspanin family comprises CD63, CD81, and CD9.

ii. Obtaining Extracellular Vesicles from Cells.

The extracellular vesicles (EVs) of the disclosure can be obtained from any cellular source. In some embodiments, the EVs are membrane (e.g., lipid bilayer) vesicles that are released from mesenchymal stromal cells. They can have, for example, a diameter ranging from about 30 nm to 1000 nm, from about 30 nm to about 500 nm, from about 50 nm to about 350 nm, or from about 30 nm to about 100 nm. In some embodiments, the isolated extracellular vesicles have a mean diameter of about 100 nm, of about 150 nm, of about 200 nm, of about 250 nm of about 300 nm, or of about 350 nm. In a preferred embodiment, the isolated extracellular vesicles have a mean diameter of about 100 nm. In another embodiment, at least 70% of the isolated extracellular vesicles have a size between 50 nm and 350 nm.

By electron microscopy, extracellular vesicles can appear to have a cup-shaped morphology. They can, for example, sediment at about 100,000×g and have a buoyant density in sucrose of about 1.10 to about 1.21 g/ml.

Mesenchymal stromal cells may be harvested from a number of sources including but not limited to bone marrow, blood, periosteum, dermis, umbilical cord blood and/or matrix (e.g., Wharton's Jelly), and placenta. Methods for harvest of mesenchymal stem cells are described in greater detail in the Examples. Reference can also be made to U.S. Pat. No. 5,486,359, which is incorporated herein by reference, for other harvest methods that can be used in the present disclosure.

The mesenchymal stromal cells, and thus the extracellular vesicles, contemplated for use in the methods of the disclosure may be obtained from the same subject to be treated (and therefore would be referred to as autologous to the subject), or they may be obtained from a different subject, preferably a subject of the same species (and therefore would be referred to as allogeneic to the subject).

As used herein, it is to be understood that aspects and embodiments of the disclosure relate to cells as well as cell populations, unless otherwise indicated. Thus, where a cell is recited, it is to be understood that a cell population is also contemplated unless otherwise indicated.

Some aspects of the disclosure refer to isolated extracellular vesicles. As used herein, an isolated extracellular vesicle is one which is physically separated from its natural environment. An isolated extracellular vesicle may be physically separated, in whole or in part, from a tissue or cellular environment in which it naturally exists, including mesenchymal stromal cells. In some embodiments of the disclosure, a composition of isolated extracellular vesicles may be free of cells such as mesenchymal stromal cells, or it may be free or substantially free of conditioned media. In some embodiments, the isolated extracellular vesicles may be provided at a higher concentration than extracellular vesicles present in un-manipulated conditioned media. Extracellular vesicles may be isolated from conditioned media from mesenchymal stromal cell culture.

Generally, any suitable method for purifying and/or enriching extracellular vesicles can be used, such as methods comprising magnetic particles, filtration, dialysis, ultracentrifugation, ExoQuick™ (Systems Biosciences, CA, USA), and/or chromatography. In some embodiments, extracellular vesicles are isolated by centrifugation and/or ultracentrifugation. Extracellular vesicles can also be purified by ultracentrifugation of clarified conditioned media. They can also be purified by ultracentrifugation into a sucrose cushion. The protocol is described in, for example, Thery et al. Current Protocols in Cell Biol. (2006) 3.22, which is incorporated herein by reference. In some embodiments, extracellular vesicles are isolated by a single step size exclusion chromatography. The protocol is described in, for example, Boing et al. Journal of Extracellular Vesicles (2014) 3:23430, which is incorporated herein by reference. A detailed method for harvest of extracellular vesicles from mesenchymal stromal cells or mesenchymal stem cells is provided in the Examples.

EVs can be used immediately or stored, whether short term or long term, such as in a cryopreserved state, prior to use. Proteinase inhibitors are typically included in freezing media as they provide extracellular vesicle integrity during long-term storage. Freezing at −20° C. is not preferable since it is associated with increased loss of extracellular vesicle activity. Quick freezing at −80° C. is more preferred as it preserves activity. See for example Kidney International (2006) 69, 1471-1476, which is incorporated herein by reference. Additives to the freezing media may be used in order to enhance preservation of extracellular vesicle biological activity. Such additives will be similar to the ones used for cryopreservation of intact cells and may include, but are not limited to DMSO, glycerol and polyethylene glycol.

iii. Synthetic EVs

A synthetic EV or exosome used according to the present embodiments can be made by different methods, as would be known to one of ordinary skill in the art. A synthetic exosome may be formed by assembling lipids into a bilayer structure (which resembles the membrane of the exosome) and functionalizing the vesicle surface with proteins, or modulating their surface by the transport of a message through direct contact with target cell receptors, or by attaching hydrophilic molecules to increase their blood circulation.

In some embodiments, the synthetic EVs or exosomes comprise liposomes. As used herein, the term “liposomes” refer to spherical vesicles having at least one lipid bilayer. Liposomes may be formed after supplying enough energy to a dispersion of lipids, such as phospholipids, in a polar solvent, such as water, to break down multilamellar aggregates into oligo- or unilamellar bilayer vesicles. Liposomes can hence be created by sonicating a dispersion of amphipathic lipids, such as phospholipids, in water.

The major types of liposomes are the multilamellar vesicle (MLV, with several lamellar phase lipid bilayers), the small unilamellar liposome vesicle (SUV, with one lipid bilayer), the large unilamellar vesicle (LUV), and the cochleate vesicle. Low shear rates create multilamellar liposomes. The original aggregates, which have many layers like an onion, thereby form progressively smaller and finally small unilamellar liposome vesicles or SUVs (which are often unstable, owing to their small size and the sonication-created defects). As an alternative to sonication, as extrusion and the Mozafari method may be employed to produce materials for human use. Using lipids other than phosphatidylcholine can greatly facilitate liposome preparation.

In particular, small unilamellar vesicles (SUVs) are ideal precursors for the preparation of vesicles that can mimic EVs due to their similarities to natural EVs (size range and membrane disposition). Thus, by applying well-known techniques used for preparation of SUV liposomes (e.g., thin-film hydration method, reverse-phase evaporation method, ethanol injection method, ether injection method, microfluidic-based methods, extrusion techniques, Mozafari, etc.), liposomes with a size range similar to that of natural EVs may be obtained.

Liposomes may be further modified to avoid detection of the body's immune system for example by coating the outside of the membrane with polyethylene glycol (PEG). Accordingly, in some embodiments the synthetic EV is coated with PEG.

### C. Method of Using the EVs of the Disclosure for Treating or Preventing Diseases or Conditions

The EVs described herein may be of particular use in treating or preventing respiratory diseases or disorders such as methods of treating or preventing acute respiratory distress syndrome (ARDS) or pulmonary fibrosis as described above. In some embodiments, the pulmonary fibrosis is idiopathic pulmonary fibrosis. In other embodiments, the EVs disclosed herein may be used to decreases systolic pulmonary arterial pressure (SPAP) in the subject, increase alveolar surface area of the lung in the subject, increase a concentration of blood oxygen in the subject, reduce extracellular matrix deposition (such as soluble collagen), improve fulton's index, or reduce inflammation in the lung in the subject.

The EVs may also be used to treat, vasculopathies such as PAH or PH; BPD; or disorders associated with mitochondrial dysfunction, reduced angiogenesis, apoptosis, or inflammation. In some embodiments, the EVs described herein may be used to alter mitochondrial function in a subject in need thereof, including a human subject. In some embodiments, the EVs described herein may increase immunomodulatory capacity of the lung. In some embodiments, the EVs described herein may reduce inflammation. In some embodiments, the EVs described herein may promote angiogenesis in the lung. In some embodiments, the EVs described herein may increase mitochondrial metabolism of the lung.

i. Bronchopulmonary Dysplasia

Bronchopulmonary Dysplasia (BPD) is a chronic lung disease of premature infants. It is characterized by prolonged lung inflammation, decrease in number of alveoli and thickened alveolar septae, abnormal vascular growth with “pruning” of distal blood vessels, and limited metabolic and antioxidant capacity. There are 14,000 new cases of BPD per year in the US. Importantly, a diagnosis of BPD often leads to other further conditions, including PAH, emphysema, asthma, increase cardiovascular morbidity and post-neonatal mortality, increased neurodevelopmental impairment and cerebral palsy, emphysema as young adults. Currently, there is no standard therapy for BPD. Some BPD patients are treated with gentle ventilation and corticosteroids, but these treatments show no effects on neuro outcomes or death. The primary risk for BPD exists in infants between 24-28 weeks after birth, which correspond to the period of the beginning of saccular development. The infants at high risk are of 1.3 to 2.2 pounds. In some embodiments, the EVs described herein may be used to treat BPD in a subject.

ii. Vasculopathy

Diseases and conditions with a vasculopathy component include, but are not limited to, pulmonary hypertension, pulmonary arterial hypertension (PAH), peripheral vascular disease (PVD), critical limb ischemia (CLI), coronary artery disease, and diabetic vasculopathy.

Pulmonary hypertension, e.g., pulmonary arterial hypertension (PAH), refers to a condition in which the pressure in the lung circulation increases, eventually causing heart failure and death. Although many causes and conditions are found to be associated with PAH, many of them share in common several fundamental pathophysiological features. One feature among these processes is dysfunction of the endothelium, the internal cellular layer of all vessel walls, which is normally responsible for the production and metabolism of a large array of substances that regulate vessel tone and repair and inhibit clot formation. In the setting of PAH, endothelial dysfunction can lead to excessive production of deleterious substances and impaired production of protective substances. Whether this is the primary event in the development of PAH or part of a downstream cascade remains unknown, but in either case, it is believed to be a factor in the progressive vasoconstriction and vascular proliferation that characterize the disease. The extracellular vesicles described herein can thus be used to treat pulmonary hypertension, including PAH.

The term peripheral vascular disease (PVD) refers to damage, dysfunction or obstruction within peripheral arteries and veins. Peripheral artery disease is the most common form of PVD. Peripheral vascular disease is the most common disease of the arteries and is a very common condition in the United States. It occurs mostly in people older than 50 years. Peripheral vascular disease is a leading cause of disability among people older than 50 years, as well as in those people with diabetes. About 10 million people in the United States have peripheral vascular disease, which translates to about 5% of people older than 50 years. The number of people with the condition is expected to grow as the population ages. Men are slightly more likely than women to have peripheral vascular disease. In some embodiments, the EVs described herein may be used to treat PVD in a subject, including a human subject.

Critical limb ischemia (CLI), due to advanced peripheral arterial occlusion, is characterized by reduced blood flow and oxygen delivery at rest, resulting in muscle pain at rest and non-healing skin ulcers or gangrene (Rissanen et al., Eur. J. Clin. Invest. 31:651-666 (2001); Dormandy and Rutherford, J. Vasc. Surg. 31:S1-S296 (2000)). Critical limb ischemia is estimated to develop in 500 to 1000 per million individuals in one year (“Second European Consensus Document on Chronic Critical Leg Ischemia”, Circulation 84(4 Suppl.) IV 1-26 (1991)). In patients with critical limb ischemia, amputation, despite its associated morbidity, mortality and functional implications, is often recommended as a solution against disabling symptoms (M. R. Tyrrell et al., Br. J. Surg. 80: 177-180 (1993); M. Eneroth et al., Int. Orthop. 16: 383-387 (1992)). There exists no optimal medical therapy for critical limb ischemia (Circulation 84(4 Suppl.): IV 1-26 (1991)). In some embodiments, the EVs described herein may be used to treat critical limb ischemia in a subject, including a human subject.

Coronary artery disease (atherosclerosis) is a progressive disease in humans wherein one or more coronary arteries gradually become occluded through the buildup of plaque. The coronary arteries of patients having this disease are often treated by balloon angioplasty or the insertion of stents to prop open the partially occluded arteries. Ultimately, these patients are required to undergo coronary artery bypass surgery at great expense and risk. In some embodiments, the EVs described herein may be used to treat coronary artery disease in a subject, including a human subject.

iii. Reduced Angiogenesis

As used herein, the term “angiogenesis” refers to the physiological process through which new blood vessels form from pre-existing vessels, formed in the earlier stage of vasculogenesis. Angiogenic activities may be evaluated by measuring endothelial tube branching points. The present disclosure shows that the EVs derived from MSC promote tube formation in human endothelial cells and prevent hyperoxia-mediated tube network loss in human endothelial cells.

In some embodiments, the EVs of the present disclosure may promote angiogenesis as determined by measuring endothelial tube branching points.

iv. Apoptosis

In preterm infants, high oxygen levels and mechanical ventilation can cause cellular stress in the lung epithelium. Oxidative stress may cause apoptosis or cell death in premature infants exhibiting respiratory distress that may lead to development of bronchopulmonary dysplasia (BPD). Oxidative stress induces mitochondrial release of cytochrome C, initiating signaling cascades leading to apoptosis. Accordingly, cellular salvage by EVs derived from MSC disclosed herein may be determined by measuring cytochrome C release.

In some embodiments, the EVs of the present disclosure may prevent apoptosis or salvage cells and tissue. In some embodiments, the EVs of the present disclosure may prevent mitochondrial release of cytochrome C, thereby preventing apoptosis. In some embodiments, the EVs of the present disclosure may treat BPD in premature infants. In some embodiments, the EVs of the present disclosure may prevent apoptosis in lung epithelium.

v. Inflammation

An inflammatory cytokine is a type of cytokine that is secreted from immune cells and certain other cell types that promote inflammation. Inflammation may be caused by cellular stress such as oxidative stress.

Inflammatory cytokines are predominantly produced by T helper cells (Th) and macrophages and involved in the upregulation of inflammatory reactions. Therapies to treat inflammatory diseases include monoclonal antibodies that either neutralize inflammatory cytokines or their receptors.

Inflammatory cytokines or chemokines may include interleukin-1 (IL-1), IL-3, IL-6 and IL-18, tumor necrosis factor alpha (TNF-α), interferon gamma (IFNγ), and granulocyte-macrophage colony stimulating factor (GM-CSF), Chemokine (C-X-C Motif) Ligand 1 (GRO), Chemokine (C-C Motif) Ligand 21 (6Ckine), Granulocyte Chemotactic Protein 2 (GCP2), or Chemokine (C-X-C Motif) Ligand 16 (CXCL16), macrophage inflammatory protein 1a (MIP1a), macrophage inflammatory protein 1b (MIP1b), interleukin 1 beta (IL1β), interleukin 12 beta (IL12β), or interferon-inducible T-cell alpha chemoattractant (ITAC). This inflammatory state of the lung is attributed to barotrauma associated with mechanical ventilation, and oxidative stress that results from high oxygen supplementation. Accordingly, the immunomodulatory activity of EVs derived from MSC may be evaluated by measuring levels of pro-inflammatory cytokines such as IL-3 or tumor necrosis factor alpha (TNF-α).

In some embodiments, the EVs of the present disclosure may prevent secretion of pro-inflammatory cytokines. In some embodiments, the pro-inflammatory cytokines comprise IL-3 or tumor necrosis factor alpha (TNF-α). In some embodiments, the EVs of the present disclosure may treat acute inflammation. EVs can enhance the presence or activation of anti-inflammatory cytokines, such as mannose receptor (CD206) and interleukin 10 (IL10). In some embodiments, the EVs of the present disclosure may treat chronic inflammation.

BPD is associated with persistent elevation of pro-inflammatory cytokines in the lung. Accordingly, the EVs of the present disclosure may be used to treat inflammation associated with BPD.

vi. Mitochondrial Dysfunction

Mitochondria are intracellular organelles responsible for a number of metabolic transformations and regulatory functions. They produce much of the ATP employed by eukaryotic cells. They are also the major source of free radicals and reactive oxygen species that cause oxidative stress. Consequently, mitochondrial defects are damaging, particularly to neural and muscle tissues, which have high energy level demands. Thus, energetic defects have been implicated in forms of movement disorders, cardiomyopathy, myopathy, blindness, and deafness (DiMauro et al. (2001) Am. J. Med. Genet. 106, 18-26; Leonard et al. (2000) Lancet. 355, 299-304). Mitochondrial dysfunction can involve increased lactate production, diminished respiration and ATP production. Mitochondrial dysfunction can manifest in oxidative stress.

Underdeveloped lungs and immature respiratory control often result in hypoxic episodes, which can lead to chronically low O2 reserves and low blood oxygen saturation in BPD patients. Improved metabolic function may be evaluated by measuring the glucose metabolism and mitochondrial oxygen consumption in pulmonary artery smooth muscle cells (PASMCs) exposed to hypoxia. In particular, the ability of the EVs to increase mitochondrial metabolism may be evaluated by measuring pyruvate kinase activity or ATPase activity.

The present disclosure provides methods for treating diseases or conditions associated with mitochondrial dysfunction. Mitochondrial dysfunction can be associated with decreased mitochondrial glucose oxidation in the subject.

In some embodiments, the disease or condition associated with mitochondrial dysfunction is selected from the group consisting of Friedreich's ataxia, Leber's Hereditary Optic Neuropathy, Kearns-Sayre Syndrome, Mitochondrial Encephalomyopathy with Lactic Acidosis and Stroke-Like Episodes, Leigh syndrome, obesity, atherosclerosis, amyotrophic lateral sclerosis, Parkinson's Disease, cancer, heart failure, myocardial infarction (MI), Alzheimer's Disease, Huntington's Disease, schizophrenia, bipolar disorder, fragile X syndrome, and chronic fatigue syndrome.

Mitochondrial Energy Production

EVs provided herein are also contemplated to be able to improve mitochondrial energy production by increasing expression of pyruvate kinase or ATPase. Cells in eukaryotic organisms require energy to carry out cellular processes. Such energy is mainly stored in the phosphate bonds of adenosine 5′-triphosphate (“ATP”). There are certain pathways that generate energy in eukaryotic organisms, including: (1) glycolysis; (2) the TCA cycle (also referred to as Krebs Cycle or citric acid cycle); and (3) oxidative phosphorylation. For ATP to be synthesized, carbohydrates are first hydrolyzed into monosaccharides (e.g., glucose), and lipids are hydrolyzed into fatty acids and glycerol. Likewise, proteins are hydrolyzed into amino acids. The energy in the chemical bonds of these hydrolyzed molecules are then released and harnessed by the cell to form ATP molecules through numerous catabolic pathways.

The main source of energy for living organisms is glucose. In breaking down glucose, the energy in the glucose molecule's chemical bonds is released and can be harnessed by the cell to form ATP molecules. The process by which this occurs consists of several stages. The first is called glycolysis, in which the glucose molecule is broken down into two smaller molecules called pyruvic acid.

In glycolysis, glucose and glycerol are metabolized to pyruvate via the glycolytic pathway. During this process, two ATP molecules are generated. Two molecules of NADH are also produced, which can be further oxidized via the electron transport chain and result in the generation of additional ATP molecules.

Glycolysis involves many enzyme-catalyzed steps that break down glucose (and other monosaccharides) into 2 pyruvate molecules. In return, the pathway leads to the generation of a sum of 2 ATP molecules. The pyruvate molecules generated from the glycolytic pathway enter the mitochondria from the cytosol. The molecules are then converted to acetyl co-enzyme A (Acetyl-CoA) for entry into the TCA cycle. The TCA cycle consists of the bonding of acetyl coenzyme-A with oxaloacetate to form citrate. The formed citrate is then broken down through a series of enzyme-catalyzed steps to generate additional ATP molecules.

Energy released from the TCA cycle in the mitochondrial matrix enters the mitochondrial electron transport chain as NADH (complex I) and FADH2 (complex II). These are the first two of five protein complexes involved in ATP production, all of which are located in the inner mitochondrial membrane. Electrons derived from NADH (by oxidation with a NADH-specific dehydrogenase) and FAD % (by oxidation with succinate dehydrogenase) travel down the respiratory chain, releasing their energy in discrete steps by driving the active transport of protons from the mitochondrial matrix to the intermembrane space (i.e., through the inner mitochondrial membrane). The electron carriers in the respiratory chain include flavins, protein-bound iron-sulfur centers, quinones, cytochromes and copper. There are two molecules that transfer electrons between complexes: coenzyme Q (complex I→III, and complex II→III) and cytochrome c (complex III→IV). The final electron acceptor in the respiratory chain is (¾, which is converted to ¾ in complex IV.

Without being bound by theory, it is believed that the EVs provided herein may increase pyruvate kinase activity and/or ATPase activity to enhance mitochondrial energy production.

### D. Assessment of the Potency of Extracellular Vesicles

As used herein, the term “potency” refers to the bioactivities of the isolated EVs.

Potency of the EVs described herein may be assessed by measuring mean linear intercept as a measurement of improved lung architecture. Measurement of mean linear intercept (MLI) is a tool used to quantify alveolar size, where a higher MLI is suggestive of reduced alveolar surface area. In some embodiments, the potent populations of extracellular vesicles are capable of decreasing MLI in a subject by at least about 10%, 15%, 20%, 25%, 30%, 35%, 40%, 45%, or 50% compared to a control left untreated with the EVs.

Potency of the EVs described herein may be assessed by measuring secretion of pro-inflammatory cytokines. In some embodiments, the potent populations of extracellular vesicles are capable of decreasing the levels of pro-inflammatory cytokines in a subject by at least about 10%, 15%, 20%, 25%, 30%, 35%, 40%, 45%, or 50% compared to a control left untreated with the EVs. The measured pro-inflammatory cytokines may for example be tumor necrosis factor alpha or interleukin-3.

Potency of the EVs described herein may be assessed by measuring mRNA expression of anti-inflammatory cytokines. In some embodiments, the potent populations of extracellular vesicles are capable of increasing the levels of anti-inflammatory cytokines in a subject by at least about 10%, 15%, 20%, 25%, 30%, 35%, 40%, 45%, 50%, 60%, 70%, 80%, 90%, 100%, 200% or more compared to a control left untreated with the EVs. The measured pro-inflammatory cytokines may for example be CD206 or interleukin-10 (IL10).

Potency of the EVs described herein may be assessed by measuring Fulton's index, which is a measure of ventricular hypertrophy and pulmonary vascular remodeling. In some embodiments, the potent populations of extracellular vesicles are capable of improving Fulton's index in a subject by at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, 200% or more compared to a control left untreated with the EVs. The measured pro-inflammatory cytokines may for example be CD206 or interleukin-10 (IL10).

Potency of the EVs described herein may be assessed by measuring secretion of cytochrome C. In some embodiments, the potent populations of extracellular vesicles are capable of decreasing the levels of cytochrome C release in a subject by at least about 10%, 15%, 20%, 25%, 30%, 35%, 40%, 45%, or 50 compared to a control left untreated with the EVs.

Potency of the EVs described herein may be assessed by measuring total branching points of blood vessels in lung tissue. In some embodiments, the potent populations of extracellular vesicles are capable of increasing the total branching points of blood vessels in lung tissue in a subject by at least about 10%, 15%, 20%, 25%, 30%, 35%, 40%, 45%, or 50% compared to a control left untreated with the EVs.

Right ventricular systolic pressure (RVSP) measurements of the effect of extracellular vesicle treatment of hypoxia induced PAH in a mouse model may be used to identify potent populations of extracellular vesicles. In some embodiments, the potent populations of extracellular vesicles are capable of reducing RVSP of mice subjected to a three-week chronic hypoxia exposure by at least about 10%, 12.5%, 15%, 17.5%, 20%, 22.5%, 25%, 27.5%, or 30%, compared to control mice subjected to a three-week chronic hypoxia exposure and treated with PBS buffer.

In some embodiments, the potent populations of extracellular vesicles may be identified by delta RVSP. As used here, delta RVSP is defined as the RVSP of hypoxia-exposed mice treated with extracellular vesicles minus RVSP of normoxia mice. In some embodiments, a population of extracellular vesicles is potent if delta RVSP is less than about 6, 5, 4, 3, or 2 mmHg.

In some embodiments, the potency of populations of extracellular vesicles may be characterized by their ability to increase O₂ consumption by smooth muscle cells (SMC) lysates. In some embodiments, the potent populations of extracellular vesicles are capable of increasing O₂ consumption by SMC lysate subjected to a 24-hour hypoxia exposure by at least about 10%, 15%, 20%, 25%, 30%, 35%, or 40%, compared to control SMC cell lysates subjected to a 24-hour hypoxia exposure and treated with PBS control.

In some embodiments, the potency of populations of extracellular vesicles may be characterized by their PK activity. In some embodiments, the potent population of extracellular vesicles have a PK activity of at least about 0.15 nmol/min/ml, 0.16 nmol/min/ml, 0.17 nmol/min/ml, 0.18 nmol/min/ml, 0.19 nmol/min/ml, 0.20 nmol/min/ml, 0.21 nmol/min/ml, 0.22 nmol/min/ml, 0.23 nmol/min/ml, 0.24 nmol/min/ml, 0.25 nmol/min/ml, 0.3 nmol/min/ml, or 0.4 nmol/min/ml.

In some embodiments, the potency of populations of extracellular vesicles may be characterized by their LDH activity. In some embodiments, the potency of populations of extracellular vesicles are characterized by their ability to decrease LDH secreted by hypoxia-exposed SMC by at least about 10%, 20%, 30%, or 40%.

In some embodiments, the isolated extracellular vesicles comprise an amount of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132 that is at least 10%, 20%, 30%, 50%, or 100% more than the average level of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132 in all extracellular vesicles of the mesenchymal stromal cells.

In some embodiments, the isolated extracellular vesicles have reduced MHCII contaminants or are substantially or totally free of MHCII contaminants, such as comprising an amount of MHCII contaminants that is at least 50%, 70%, 80%, 90%, 95%, 98%, or 99% less than the average level of MHCII contaminants in all extracellular vesicles of the mesenchymal stromal cells.

In some embodiments, the isolated extracellular vesicles have reduced fibronectin content or are substantially or totally free of fibronectin, such as comprising an amount of fibronectin that is at least 50%, 70%, 80%, 90%, 95%, 98%, or 99% less than the average level of fibronectin in all extracellular vesicles of the mesenchymal stromal cells.

### E. Treatment Using Extracellular Vesicles

Compositions useful for the methods of the present disclosure can be administered via, inter alia, localized injection, including catheter administration, systemic injection, localized injection, intravenous injection, intrauterine injection or parenteral administration. When administering a therapeutic composition described herein (e.g., a pharmaceutical composition), it will generally be formulated in a unit dosage injectable form (e.g. solution, suspension, or emulsion).

In any of the embodiments, there may be single or repeated administration of extracellular vesicles, including two, three, four, five or more administrations of extracellular vesicles. In some embodiments, the isolated EV is administered in 2 doses, 3 doses, 4 doses, 5 doses, 6 doses, 7, doses, 8 doses, 9 doses, 12 doses, 15 doses, 18 doses, or more. In some embodiments, the isolated EV is administered in 2 doses, 3 doses, 4 doses, 5 doses, 6 doses, 7, doses, 8 doses, 9 doses, 12 doses, 15 doses, 18 doses, or more within a week. In some embodiments, the extracellular vesicles may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks) depending on the severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). In some embodiments, the isolated EV is administered at an interval of 12 hours, 24 hours, 48 hours, 72 hours, 4 days, 5, days, 6 days, or once per week. In some embodiments, the isolated EV is administered once daily for 2 days, for 3 days, for 4 days, for 5 days, or for a week The time between administrations may be the same or they may differ. As an example, if the symptoms of the disease appear to be worsening the extracellular vesicles may be administered more frequently, and then once the symptoms are stabilized or diminishing the extracellular vesicles may be administered less frequently. In some embodiments, the EVs can be administered upon onset of respiratory distress, such as ARDS, and can continue to be administered for at least the duration of the respiratory distress. In some embodiments, the EVs can be administered for most or all of the duration of mechanical ventilation. Such administration may reduce inflammation, either resulting from the underlying condition or the mechanical ventilation itself. Such administration may reduce deleterious effects of the mechanical ventilation.

EVs can be administered repeatedly in low dosage forms or as single administrations of high dosage forms. Low dosage forms may range from, without limitation, 1-50 micrograms per kilogram, while high dosage forms may range from, without limitation, 51-1000 micrograms per kilogram. It will be understood that, depending on the severity of the disease, the health of the subject, and the route of administration, inter alia, the single or repeated administration of low or high dose extracellular vesicles are contemplated.

The unit dose of EV may be phospholipids of EVs per kg of subject being treated. In some embodiments, the effective dose of the isolated EV is 50 pmol of phospholipids of EVs per kg of subject being treated (pmol/kg). In some embodiments, the effective dose of the isolated EV is from 20 to 500 pmol of phospholipids of EVs per kg of subject being treated (pmol/kg). In some embodiments, the effective dose of the isolated EV is from 100 to 500 pmol of phospholipids of EVs per kg of subject being treated (pmol/kg). In some embodiments, the effective dose of the isolated EV is from 200 to 500 pmol of phospholipids of EVs per kg of subject being treated (pmol/kg). In some embodiment, the effective dose of the isolated EV is between 20-150 pmol/kg. In some embodiment, the effective dose of the isolated EV is between 25-100 pmol/kg. In some embodiment, the effective dose of the isolated EV is between 25-75 pmol/kg. In some embodiment, the effective dose of the isolated EV is between 40-60 pmol/kg. of phospholipids of EVs per kg of subject being treated.

The EVs may be used in combination treatments. In some embodiments, the EVs are administered with a therapeutic agent comprising one or more of a phosphodiesterase type-5 (PDE5) inhibitor, a prostacyclin agonist, or an endothelin receptor antagonist. In some embodiments, wherein the isolated EV and the therapeutic agent are administered in the same composition. In some embodiments, the EVs and the therapeutic agent are administered in separate compositions, substantially simultaneously or sequentially. In some embodiments, the isolated EV and therapeutic agent are administered at an interval of 6 hours, 12, hours, 24 hours, 48 hours, 72 hours, 4 days, 5, days, 6 days, or once per week.

In some embodiments, the method further comprises administering a phosphodiesterase type-5 (PDE5) inhibitor as the therapeutic agent. In some embodiments, the PDE5 inhibitor comprises sildenafil, vardenafil, zapravist, udenafil, dasantafil, avanafil, mirodenafil, or lodenafil. In some embodiments, the PDE5 inhibitor is sildenafil. In some embodiments, the isolated EV and the phosphodiesterase type-5 (PDE5) inhibitor are administered in separate compositions, substantially simultaneously or sequentially. In some embodiments, the isolated EV and the phosphodiesterase type-5 (PDE5) inhibitor are administered in the same composition. In some embodiments, the isolated EV and the PDE5 inhibitor are administered in one or more doses. In some embodiments, the isolated EV and PDE5 inhibitor are administered at an interval of 6 hours, 12, hours, 24 hours, 48 hours, 72 hours, 4 days, 5, days, 6 days, or once per week. In some embodiments, the isolated EV is administered in 2 doses, 3 doses, 4 doses, 5 doses, 6 doses, 7 doses, 8 doses, 9 doses, 12 doses, 15 doses, 18 doses, or more, and wherein the PDE5 inhibitor is administered in 16 doses, 19 doses, 21 doses, 24 doses, 27 doses, 30 doses, 33 doses, 36 doses, 39 doses, 42 doses, 45 doses, 48 doses, 51 doses, 54 doses, 57 doses, 60 doses, 63 doses, 66 doses, or more. In some embodiments, the isolated EV is administered in 2 doses, 3 doses, 4 doses, 5 doses, 6 doses, 7 doses, 8 doses, 9 doses, 12 doses, 15 doses, 18 doses, or more within a week, and wherein the PDE5 inhibitor is administered in 16 doses, 19 doses, 21 doses, 24 doses, 27 doses, 30 doses, 33 doses, 36 doses, 39 doses, 42 doses, 45 doses, 48 doses, 51 doses, 54 doses, 57 doses, 60 doses, 63 doses, 66 doses, or more within a week.

In some embodiments, the EVs may be administered with a prostacyclin agonist. In some embodiments, the prostacyclin agonist comprises epoprostenol sodium, treprostinil, beraprost, ilprost, and a PGI2 receptor agonist. In some embodiments, the isolated EV and the prostacyclin agonist are administered at an interval of 6 hours, 12, hours, 24 hours, 48 hours, 72 hours, 4 days, 5, days, 6 days, or once per week. In some embodiments, the isolated EV and the prostacyclin agonist are administered in one or more doses. In some embodiments, the isolated EV and the prostacyclin agonist are administered in separate compositions, substantially simultaneously or sequentially. In some embodiments, the isolated EV and the prostacyclin agonist are administered in the same composition.

In some embodiments, the EV may be administered with a endothelin receptor agonist. In some embodiments, the isolated EV and the endothelin receptor agonist are administered in one or more doses. In some embodiments, the isolated EV and the endothelin receptor are administered at an interval of 6 hours, 12, hours, 24 hours, 48 hours, 72 hours, 4 days, 5, days, 6 days, or once per week. In some embodiments, the isolated EV and the endothelin receptor agonist are administered in separate compositions, substantially simultaneously or sequentially. In some embodiments, the isolated EV and the endothelin receptor agonist are administered in the same composition.

The extracellular vesicles may be used (e.g., administered) in pharmaceutically acceptable preparations (or pharmaceutically acceptable compositions), typically when combined with a pharmaceutically acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, or solvent encapsulating material.

Such preparations may routinely contain pharmaceutically acceptable concentrations of salt, buffering agents, preservatives, compatible carriers, and may optionally comprise other (i.e., secondary) therapeutic agents. A pharmaceutically acceptable carrier is a pharmaceutically acceptable material, composition or vehicle, such as a liquid or solid filler, diluent, excipient, solvent or encapsulating material, involved in carrying or transporting a prophylactically or therapeutically active agent. Each carrier must be “acceptable” in the sense of being compatible with the other ingredients of the formulation and not injurious to the subject. Some examples of materials which can serve as pharmaceutically acceptable carriers include sugars, such as lactose, glucose and sucrose; salts such as sodium chloride; ethylenediaminetetraacetic acid (EDTA); glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other nontoxic compatible substances employed in pharmaceutical formulations.

The preparations are administered in effective amounts. An effective amount is that amount of an agent that alone stimulates the desired outcome. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

Other embodiments include a packaged and labelled pharmaceutical product. This article of manufacture or kit includes the appropriate unit dosage form in an appropriate vessel or container such as a glass vial or plastic ampoule or other container that is hermetically sealed. The unit dosage form should be suitable for pulmonary delivery for example by aerosol. Preferably, the article of manufacture or kit further comprises instructions on how to use including how to administer the pharmaceutical product. The instructions may further contain informational material that advises a medical practitioner, technician or subject on how to appropriately prevent or treat the disease or disorder in question. In other words, the article of manufacture includes instructions indicating or suggesting a dosing regimen for use including but not limited to actual doses, monitoring procedures, and other monitoring information.

As with any pharmaceutical product, the packaging material and container are designed to protect the stability of the product during storage and shipment. The kits may include MSC extracellular vesicles in sterile aqueous suspensions that may be used directly or may be diluted with normal saline for intravenous injection or use in a nebulizer, or dilution or combination with surfactant for intratracheal administration. The kits may therefore also contain the diluent solution or agent, such as saline or surfactant.

### EXAMPLES

The following examples are intended to provide those of ordinary skill in the art with a complete disclosure and description of how to make and use the methods and compositions described herein, and are not intended to be limiting.

### Example 1.1—General Methods of Isolating EV Populations

This example demonstrates isolation of EVs from a cell culture media.

Filtration: Conditioned media obtained from mesenchymal stem cells (MSCs) was pumped through a filter line to eliminate any cells, dead cells, and cellular debris. Then, the condition media was supplemented with 25 mM HEPES and 10 mM EDTA buffers.

Tangential flow filtration: the conditioned media was concentrated by tangential flow filtration (TFF) system with a single 100 kDa MWCO cassette. The retentate was collected and filtered using a 0.22 um filter. The filtrate was divided into 10 mL aliquot sample and frozen at −80° C.

Diafiltration: Samples may be optionally subjected to a diafiltration step, preferably after the TFF step and before the Fractionation step which is similar to buffer exchange. Once a desired concentration of EVs is reached, PBS buffer was added to the sample through a reservoir to maintain the volume while continuing to run the pump to the TFF cassette filter. Gradually, the PBS replaced the conditioned media. In order to achieve as complete of an exchange as possible, 7 total volume diafiltrations were performed to with the retentate. This step helps to remove some of the impurities in the retentate, without affecting EVs. The presence of EVs was verified by FLOT-1 western blots, which shows decreased amount of total protein and phospholipid.

Fractionation: Samples were thawed at 37° C. for approximately 10 minutes. All samples were pooled together in a 150 ml corning bottle. Axichrom 70/500 column was packed with Sepharose CL-2B resin (GE). The Axichrom 70/500 column is connected to an AKTA Avant 150 (GE). The sample was introduced into the column via the sample line. Once all the sample was introduced to the column, the elution step began (settings: flow rate of 9.0 ml/min). 0.2 column volumes (CV) of void column eluted out and then the fraction collector started collecting fractions at a rate of 1 minute per fraction (4.6 ml in each fraction). Fractions were collected until 0.6 CV was eluted out (EVs eluted out between 0.3 CV-0.4 CV). PBS was used for the entire experiment. The fraction samples are capped under the hood and stored at 4° C.

Measuring phospholipid concentration: Phospholipid signaling was used for EV detection. Briefly, after fractionation, 20 uL of each EV prep and 80 uL of a reaction mix (Sigma) were transferred into black, clear-bottom 96-well plates (Corning, Corning, N.Y.) and incubated for 30 minutes at room temperature protected from light. Fluorescence intensity was measured at 530/585 nm using a FLUOstar™ Omega microplate reader (BMG Labtech, Ortenberg, Germany). In the EV production runs shown, both A214 chromatograms and phospholipid were utilized for EV detection.

### Example 1.2—Characterization of EVs Isolated Using Size Exclusion Chromatography from Mesenchymal Stem Cells (MSC)

This example demonstrates isolation of EVs form MSC and that EVs isolated from MSC can be distinguished from fibroblast-derived EVs by chromatographic profiles.

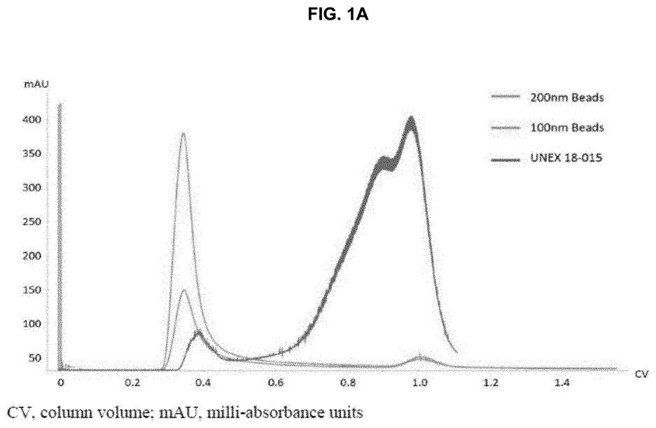

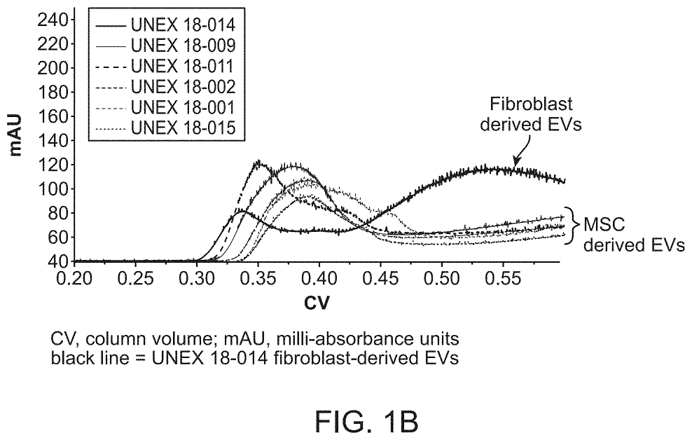

EVs were purified from cell culture supernatant from mesenchymal stem cells (MSCs) using size exclusion chromatography (SEC) with a Sepharose®-based resin, which separates the extracellular vesicles (EVs) from other cell-secreted factors. The drug substance batches comprising the purified EVs from MSCs are referred to as UNEX 18-001, UNEX 18-002, UNEX 18-009, UNEX 18-011, and UNEX 18-015, and these development grade batches of EVs are collectively referred to as UNEX-42. For comparison, EVs were also isolated from fibroblast cell cultures, and a batch comprising fibroblast-derived EVs is referred to as UNEX-18-014. During this purification step, an in-process chromatographic profile was generated based on ultraviolet (UV) light absorbance at 214 nm (A214) as the material elutes from the column. As shown in FIG. 1, the EV bulk drug substance coincides with the absorbance peak that is observed between approximately 0.3 and 0.5 column volumes (CVs). Reference polystyrene beads with diameters of 100 nm (Phosphorex, 4002) and 200 nm (Phosphorex, 2202) were run under identical chromatography conditions, and their profiles overlaid with UNEX 18-015 as the representative purification chromatogram as shown in FIG. 1A While the current SEC resin and parameters cannot resolve the difference between 100 nm and 200 nm, these data confirm that the particle size of the isolated EV approximates the size range of these reference beads.

The chromatographic profiles of EV batches isolated from MSCs using SEC were overlaid and compared to fibroblast-derived EVs as shown in FIG. 1B. These data show the consistency of the EV purification runs based on elution volume, peak magnitude and overall shape/asymmetry of the curve. Importantly, the chromatograms of the EV reveal a profile that is distinct from fibroblast-derived EVs.

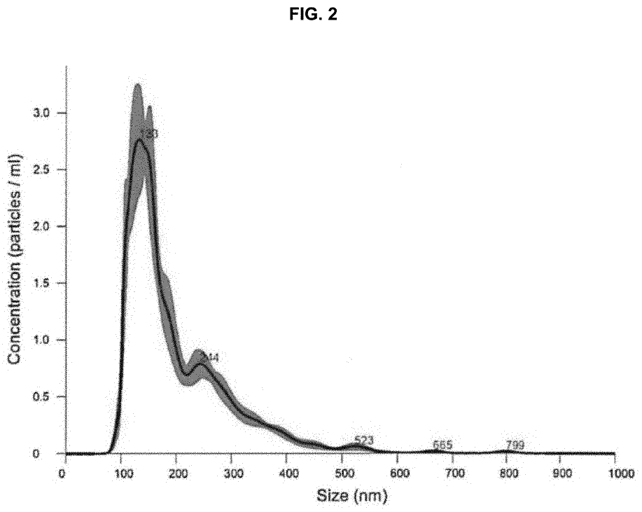

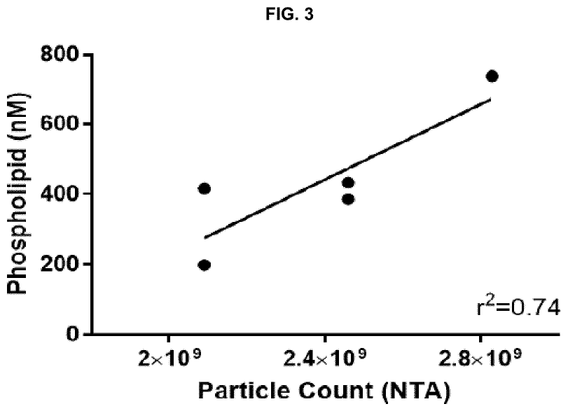

Nanoparticle tracking analysis (NTA) is a video-based method that determines particle size and concentration based on light scatter and real-time particle movements. NTA is a commonly used tool to determine EV quantity and size distribution in a solution. Using established NTA methods which generate distribution histograms, development-grade batches of EVs were found to contain particles primarily in the size range from approximately 50 to 350 nm in diameter as shown in FIG. 2 and Table 1 below. Consistent with published findings on MSC-derived EV and based on the empirical data presented in FIG. 2, the herein isolated EVs from MSCs represent a highly polydisperse population. Metrics that are captured to describe this distribution profile include (1) the weighted mean, (2) the weighted mode, and (3) the size at which the measured particles fall below the 10th, 50th, and 90th percentile of the entire population. The size distribution, as well as the particle concentration, are similar among development-grade batches of mesenchymal stem cell derived EVs and fibroblast-derived EVs as shown in Table 1 below.

TABLE 1
Nanoparticle Tracking Analysis on EVs derived from MSCs and
fibroblast-derived EVs.

| In-house Batch | Particle Distribution (nm)^{a} | | | Weighted | Weighted | Particles |
|---|---|---|---|---|---|---|
| Number | D[n, 0.10] | D[n, 0.50] | D[n, 0.90] | Mean (nm) | Mode (nm) | per mL |
| UNEX 18-001 | 157.7 | 257.5 | 427.5 | 281.5 | 199.4 | 2.08 × 10⁹ |
| UNEX 18-002 | 118.2 | 170.5 | 334.3 | 208.7 | 128.6 | 2.46 × 10⁹ |
| UNEX 18-009 | 124.7 | 197.6 | 373.7 | 229.9 | 161.4 | 2.46 × 10⁹ |
| UNEX 18-011 | 120.2 | 170.1 | 303.8 | 198.8 | 137.3 | 2.83 × 10⁹ |
| UNEX 18-015 | 114.5 | 164.0 | 329.2 | 200.8 | 200.8 | 2.04 × 10⁹ |
| UNEX 18-014 | 101.3 | 190.6 | 392.9 | 224.2 | 122.8 | 8.71 × 10⁸ |
| (Fibroblast EV) | | | | | | |

^{a}Particle distribution is expressed as D[n, 0.##] which is the size at which the popluation falls below the ## percentile.

### Example 1.4—Global Proteomic Analysis to Determine the Protein Profile of EVs Derived from MSCs

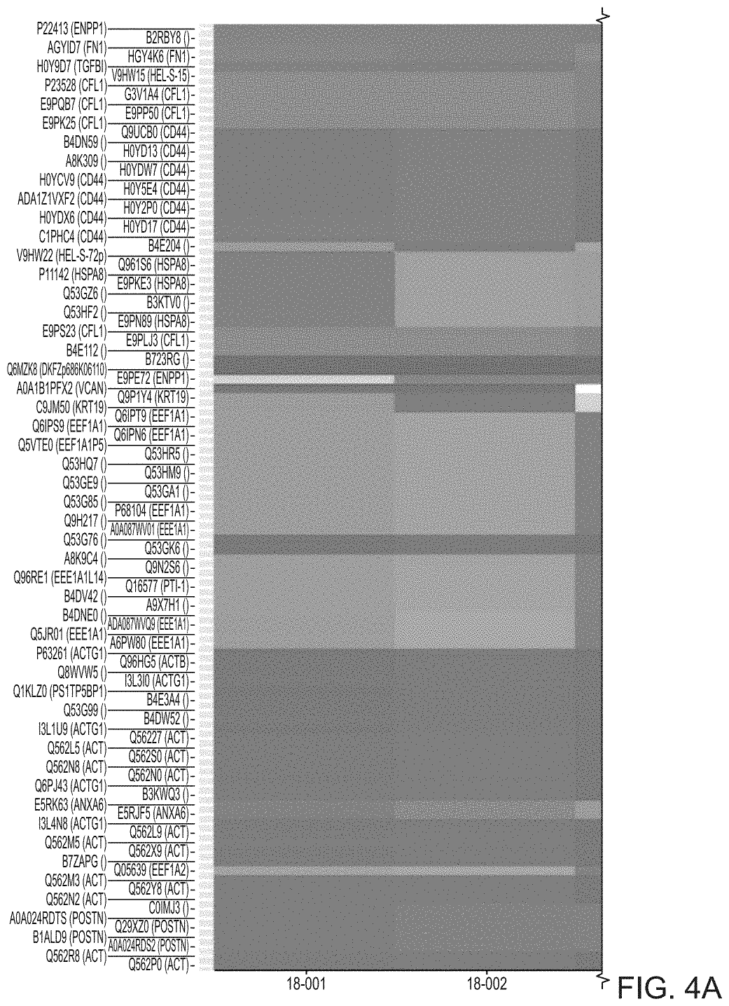

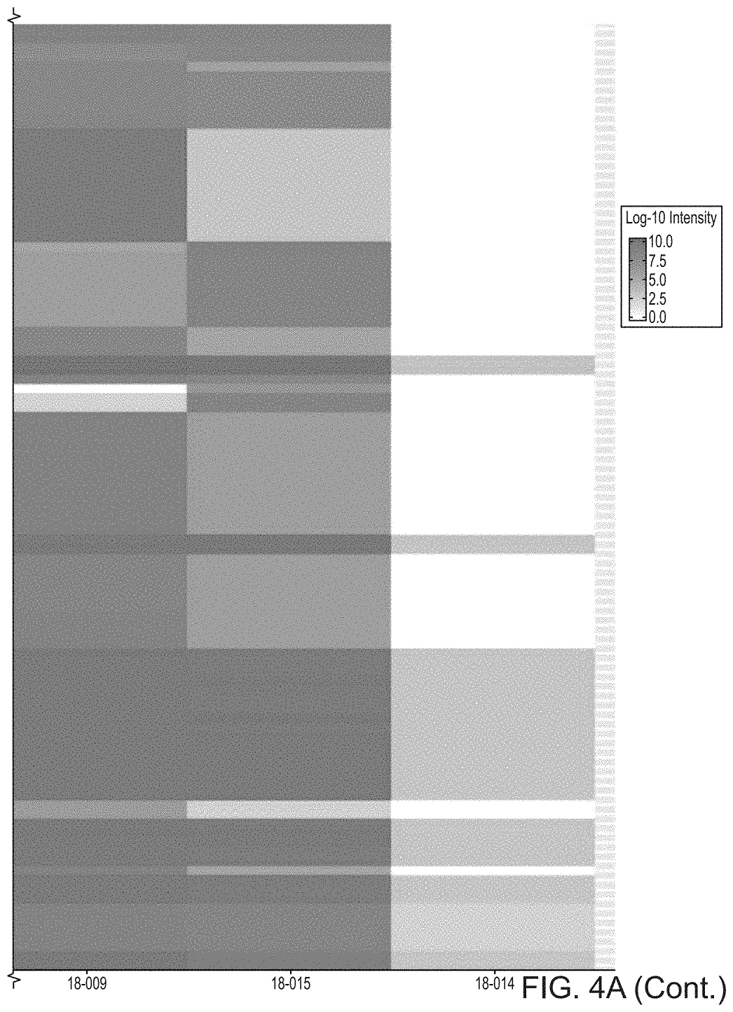

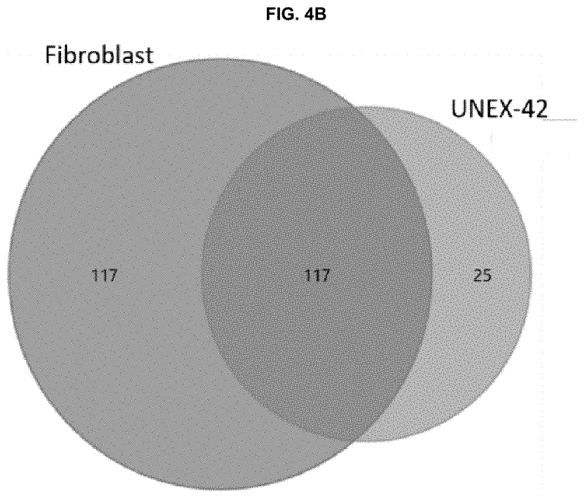

Global proteomics analysis was conducted to generate protein profiles for each of the development-grade batches of EVs derived from MSCs. Profiles were compared among batches to determine product consistency, and they were also compared to the profile of fibroblast-derived EVs to identify the specific protein profile of EVs derived from MSCs. Each sample was submitted in triplicate for trypsin digestion followed by peptide profiling by UPLC-MS/MS. Batch UNEX 18-011 was excluded from analysis due to insufficient reads. Based on these data, a heat map was generated showing the top 100 differentially contained proteins between batches of MSC-derived EVs and fibroblast-derived EVs as presented in FIG. 4A. These profiles indicate that there is high similarity among the batches of MSC-derived EVs, and their content differs from the protein profile of fibroblast-derived EVs. Additional analysis was conducted to determine the most common proteins among batches of MSC-derived EVs.

Importantly, 142 sequences were identified as being present in all 4 batches of MSC-derived EVs that were analyzed. The 142 hits were compared to fibroblast-derived EVs, and a set of 25 proteins specific for MSC-derived EVs were identified (FIG. 4B), with a complete list grouped by cellular function presented in Table 3 below.

TABLE 3
Functional Classes of proteins specific for MSC-derived EVs.

| Protein Functional Class | List of Proteins in UNEX-42 Only |
|---|---|
| Cytoskeletal | KRT19, TUBB, TUBB2A, TUBB2B, |
| | TUBB2C, TUBB3, TUBB4B, TUBB6, CFL1 |
| | (HEL-S-15), VIM |
| Gene Transcription/ | EEF1A1, EEF1A1P5, PTI-1, EEF1A1L14, |
| Translation | EEFA2 |
| Nucleases, Nucleotidases | ENPP1, NT5E |
| Heat Shock Proteins | HSPA8 (HEL-S-72p) |
| Vesicle Trafficking | RAB10 |
| ECM Interactions | CD44 |
| Proteolysis | MMP2 |
| Cell Signaling | CD109 |
| Unknown | DKFZp686P132 |

### Example 1.5—Analysis of Specific Markers for Development Grade Batches of EVs

EV preparations should be minimally characterized based on the semi-quantitative or quantitative assessment of at least 3 expected proteins according to the International Society for Extracellular Vesicles (ISEV). The expected proteins may be: 1) located within the vesicle lumen, 2) associated with moieties on the vesicle surface, or 3) embedded in the lipid bilayer through hydrophobic domains. Moreover, it is recommended that EV preparations should be assessed for the absence of proteins that are not expected to be enriched in the population of interest. Using semi-quantitative (e.g., Western blot) or quantitative (e.g., enzyme-linked immunosorbent assay [ELISA]) protein assay methods, the MSC derived EV development-grade preparations (referred to as UNEX-42) were analyzed for the presence of expected and unexpected proteins of interest. Individual UNEX-42 batches are referred to as UNEX 18-001, UNEX 18-002, UNEX 18-009, UNEX 18-011, and UNEX 18-015 in Table 4 that presents the results of the marker analysis. These results were compared to a fibroblast-derived EV batch referred to as UNEX 18-014.

TABLE 4
Qualitative and Quantitative protein analysis of EVs
derived from MSC.

| In-house | Intra- | | Transmembrane | | | |
|---|---|---|---|---|---|---|
| Batch | luminal | Surface | FLOT- | CD105^{a,b} | MHC- | MHC- |
| Number | SDCBP | ANXA2 | 1^{b} | (ng/mL) | I | II |
| UNEX_18-001 | (+) | (+) | (+) | 0.47 | (+) | ND |
| UNEX_18-002 | (+) | (+) | (+) | 1.19 | (+) | ND |
| UNEX_18-009 | (+) | (+) | (+) | 0.90 | (+) | ND |
| UNEX_18-011 | (+) | (+) | (+) | 0.72 | (+) | ND |
| UNEX_18-015 | (+) | (+) | (+) | 0.33 | (+) | ND |
| UNEX_18-014 | ND | (+) | (+) | <0.2 | ND | ND |
| (Fibroblast EV) | | | | | | |

(+), detected by Western blot: ANXA2, annexin A2; CD, cluster of differentiation; EV, extracellular vesicles; FLOT-1, flotillin 1; MHC-I major histocompatibility complex class I; MHC-II major histocompatibility complex class II; ND, not detected; SDCBP, syndecan binding protein or syntenin-1
^{a}Protein selected for UNEX-42 drug substance specification
^{b}Protein seleced for UNEX-42 drug product specification

Table 4 presented amount the amounts of Syntenin-1 (or syndecan binding protein, SDCBP), Anxa2, Flotillin (FLOT-1, CD105, MHC-I, and MHC-II in the UNEX-42 batches as compared to the fibroblast-derived batch.

Syntenin-1 (or syndecan binding protein, SDCBP) is an adapter protein involved in transmembrane protein trafficking, vesicle sorting, and exosome biogenesis. Syntenin-1 associates indirectly with components of the endosomal-sorting complex required for transport (ESCRT) machinery and directly with the tetraspanin class of transmembrane proteins. Syntenin-1 was detected in all 5 UNEX-42 development-grade batches of EVs derived from MSC by Western blot, but not in fibroblast-derived EVs as presented in Table 4.

Annexins are a family of proteins that are characterized by their calcium-dependent phospholipid-binding properties and play a role in organizing membrane lipid domains. Annexin A2 can be localized to both the outer and inner leaflets of EVs, and has been shown to associate with phosphatidylserine lipid rafts and cholesterol microdomains. Annexin A2 was detected by Western blot in all UNEX-42 development-grade batches of EVs derived from MSC, as well as in fibroblast-derived EVs as presented in Table 4.

Flotillin 1 (FLOT-1) is a membrane-associated protein involved in endocytosis, and endosomal trafficking. Flotillin 1 is a well-established protein marker for EVs, and was detected by Western blot in all UNEX-42 development-grade batches of EVs derived from MSC, as well as in fibroblast-derived EVs (Table 4). Detection of flotillin-1 has been implemented for release testing of UNEX-42 drug product.

CD105 (or endoglin, ENG) is an accessory receptor for the transforming growth factor beta (TGFβ) superfamily of signaling molecules, and is expressed on the plasma membrane surface of MSC. EVs are known to harbor similar surface marker profiles as their cells of origin, and MSC-derived EVs have previously been shown to contain CD105. Protein levels of CD105 were evaluated on development-grade batches of UNEX-42 and fibroblast-derived EVs by ELISA with results listed in Table 4. CD105 was detected in all 5 UNEX-42 development-grade batches of EVs derived from MSC, but not in fibroblast-derived EVs (Table 4). The CD105 ELISA method has been implemented as an identity marker for both UNEX-42 drug substance and UNEX-42 drug product release testing.

Major histocompatibility complex class I (MHC-I) proteins, which include human leukocyte antigens (HLA-) A, B, and C, are cell surface proteins that facilitate cytosolic antigen presentation to cytotoxic T lymphocytes. MSCs are known to express variable levels of MHC-I proteins, and by extension, MHC-I proteins were expected to be present on bone marrow MSC-derived EVs. All 5 UNEX-42 development-grade batches of EVs derived from MSC were found to express MHC-I, but MHC-I was not detected in fibroblast-derived EVs (Table 4).

Major histocompatibility complex class II (MHC-II) proteins, which includes the HLA-DR proteins and others, facilitate exogenous antigen presentation to helper T lymphocytes. Unlike MHC-I, MSC do not express MHC-II, which conveys some level of immune-privilege in the context of allogeneic transplantation. As expected, UNEX-42 batches of EVs derived from MSC does not express MHC-II, nor is it detected in fibroblast-derived EVs (Table 4).

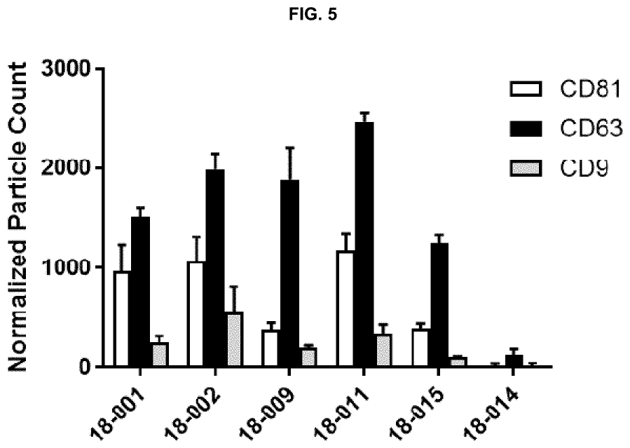

The UNEX-42 batches of EVs derived from MSC were also characterized for expression of members of the tetraspanin family of transmembrane proteins are involved in membrane organization, endosome trafficking, and extracellular vesicle biogenesis. CD63, CD81, and CD9 are the tetraspanin proteins most commonly associated with extracellular vesicles. The presence and frequency of CD63, CD81, and CD9 was determined using the ExoView™ analysis platform (NanoView Biosciences), which combines features of immunoaffinity microarrays with enhanced light-scattering microscopy. Briefly, EVs were first immobilized on a chip array using capture antibodies of interest, specifically CD63, CD81, and CD9. Bound particles were visualized under enhanced brightfield and were counted using the platform software. A summary of the data is presented in FIG. 5. CD63 was the predominant surface marker in all 5 UNEX-42 development-grade batches of EVs derived from MSC. UNEX-42 development-grade batches of EVs derived from MSC contained moderate amounts of CD81 and low amounts of CD9. All 3 tetraspanins were minimally detected in fibroblast-derived EVs.

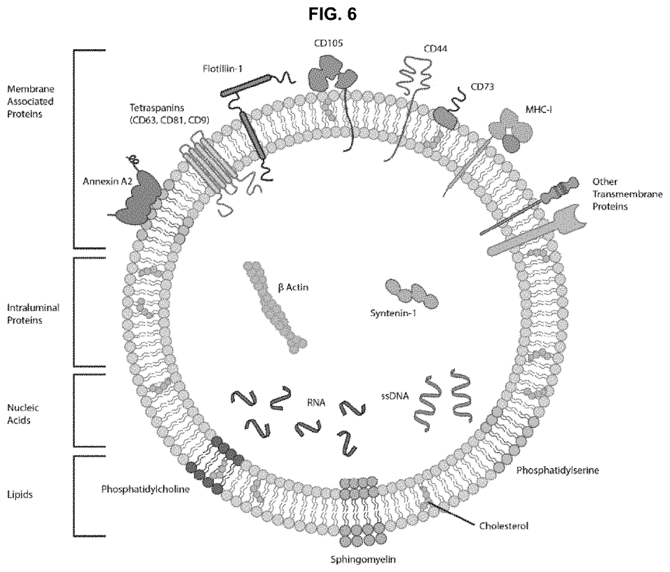

FIG. 6 shows an exemplary generalized schematic of the UNEX-42 EVs derived from MSC based on the data presented above.

### Example 1.6—Angiogenic Activities of the EVs Derived from MSC

The EVs derived from MSC were shown to promote tube formation in human endothelial cells, and to prevent hyperoxia-mediated tube network loss in human endothelial cells.

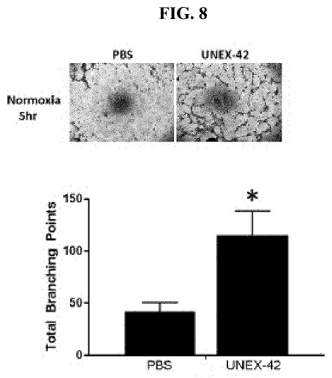

To assess the ability of UNEX-42 to promote angiogenesis, human umbilical vein endothelial cells (HUVECs) were grown on Matrigel-coated plates to roughly 80% confluence. After a 3-hour pre-treatment with PBS or UNEX-42, endothelial tube branching points were evaluated over 5 hours. As shown in FIG. 8, UNEX-42 increased total branching points more than 2-fold, suggesting that UNEX-42 may promote microvascular network formation in infants at risk for developing bronchopulmonary dysplasia (BPD).

Moreover, HPAECs exposed to normal air were allowed to form networks in culture. Cells were then exposed to PBS or UNEX-42 for 3 hours prior to exposure to normoxia (21% O2) or hyperoxia (97% O2) for 40 hours to model hyperoxia-mediated vascular network damage. Tube branching points were evaluated, and exposure of control cells to hyperoxia resulted in a deterioration of the HPAEC network, as indicated by a reduction in branching points, whereas UNEX-42 pre-treatment fully prevented this deterioration as shown in FIG. 9.

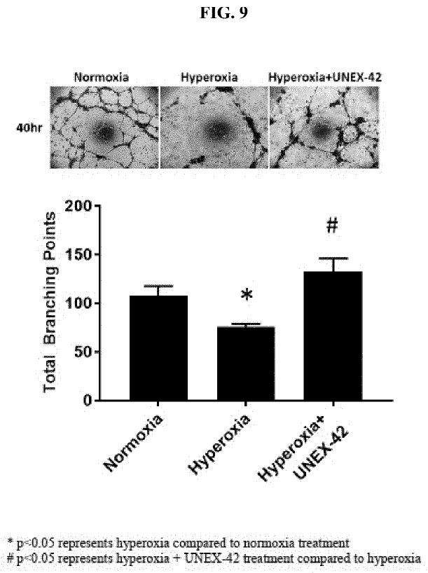

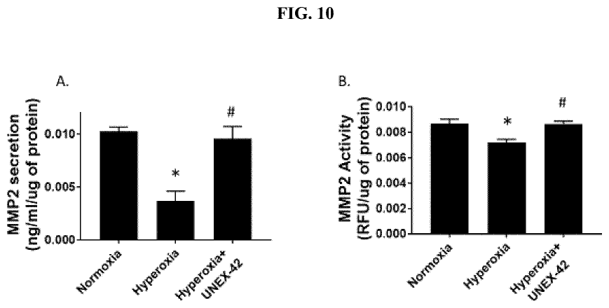

Moreover, it was tested whether the herein disclosed EVs derived from MSC can prevent reduction of matrix metalloproteinases (MMPs) required for normal lung development. To assess whether the herein disclosed EVs derived from MSC can prevent reduction of MMP2 levels, HUVECs were exposed to PBS or UNEX-42 for 3 hours prior to 24 hours of normoxia or hyperoxia (97% O2), followed by measuring the amount of MMP2 secreted into the media by using enzyme-linked immunosorbent assay (ELISA). As shown in FIG. 10, MMP-2 levels were reduced after hyperoxia exposure, which was prevented by UNEX-42 pre-treatment.

These results indicate that the herein disclosed EVs derived from MSC have angiogenic activities. Moreover, in some embodiments, the herein disclosed EVs derived from MSC may be used for treating BDP by promoting angiogenesis and protecting existing blood vessels from high oxygen treatment in infants with BPD.

### Example 1.7—Cellular Salvage by EVs Derived from MSC

The purpose of this example was to test if the EVs derived from MSC can be used as treatment to prevent bronchopulmonary dysplasia (BPD) in premature infants exhibiting respiratory distress by preventing release of cytochrome C caused by the oxidative stress. To model the lung epithelial cell damage seen in BPD, an in vitro study was conducted using A549 cells.

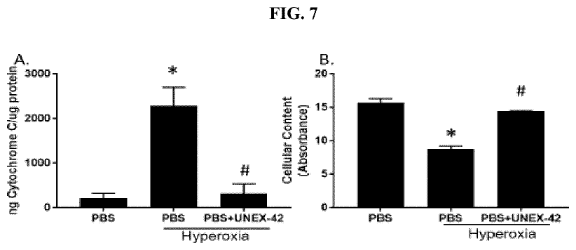

Hyperoxia exposure of A549 cells led to a dramatic induction of secreted cytochrome C and a loss of viable cells as measured by total nucleic acid content remaining in the culture, whereas UNEX-42 EVs prevented both effects, maintaining normal cytochrome C levels and reducing viable cell loss as shown in FIG. 7. In particular, FIG. 7 showed that UNEX-42 EVs reduced the hyperoxia induced release of cytochrome C (A) and maintained the cellular levels of cytochrome C in hyperoxia treated cells to that of normoxic cells (B).

Accordingly, the EVs derived from MSC were shown to prevent secretion of cytochrome C and to reduce viable cell loss in A549 lung carcinoma cells exposed to hyperoxia.

These results indicate that the herein disclosed EVs derived from MSC salvage cell viability, and may be used as treatment to prevent bronchopulmonary dysplasia (BPD) in premature infants exhibiting respiratory distress by preventing release of cytochrome C caused by the oxidative stress.

### Example 1.8—Improved Metabolic Function by EVs Derived from MSC

The EVs derived from MSC were shown to increase glucose metabolism and improve mitochondrial oxygen consumption in pulmonary artery smooth muscle cells (PASMCs) exposed to hypoxia.

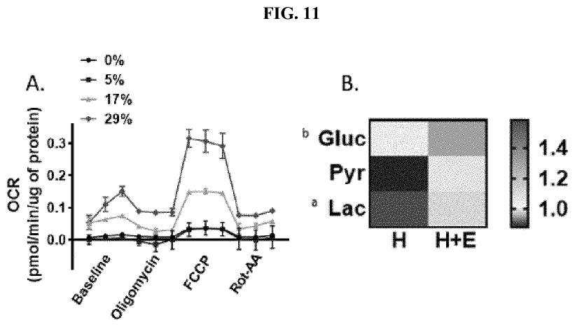

To assess effect of EVs derived from MSC on oxygen consumption, PASMCs were exposed to 24 hours of hypoxia, followed by a mitochondrial stress test whereby a series of compounds were injected into the cell culture system to determine ATP production (oligomycin), maximal respiration (carbonyl cyanide p-triflouromethoxyphenylhydrazone [FCCP]), and non-mitochondrial respiration (rotenone/antimycin A). UNEX-42 increased oxygen consumption of hypoxic cells in a dose-dependent manner during all phases of the mitochondrial stress test as shown in FIG. 11A. These data demonstrate the potential of UNEX-42 to increase oxygen consumption after hypoxic exposure.

Further experiments investigated the metabolites involved in this mitochondrial advantage. PASMCs were cultured to confluence and cells were treated with PBS or UNEX-42. Cells were maintained in hypoxia (4% oxygen) for 2 weeks, treated biweekly on culture days 1, 4, 8, and 11 with either UNEX-42 or PBS. Metabolite analysis was done using a global metabolomics platform which utilizes ultra high-performance liquid chromatography/tandem accurate mass spectrometry (UHPLC/MS/MS) and a mLIMS metabolite standard library for metabolomics profiling. Metabolomics data was analyzed using Metabolync software (Metabolon, Morrisville, N.C.). Hypoxia resulted in a dramatic conversion of pyruvate into lactate, resulting in high lactate levels in the culture media consistent with the glycolytic shift seen in PAH as shown in FIG. 11B. Addition of UNEX-42 reduced levels of glucose in the culture media, indicating an increase in glucose uptake. Addition of UNEX-42 also resulted in a reduction in lactate levels in the culture media, indicating pyruvate entry into the mitochondria, thereby decreasing lactate production FIG. 11B. This combined effect is reflective of an increase in nutrient flux into the mitochondria during chronic hypoxia exposure and supports a therapeutic benefit of UNEX-42 in the context of both BPD and BPD-associated PH.

### Example 1.9—Immunomodulatory Activities of the EVs Derived from MSC

The EVs derived from MSC was shown to prevent cytokine secretion in A549 lung carcinoma cells exposed to hyperoxia, and to reduce cytokine and chemokine secretion in vitro in THP1 monocytic leukemia cells after lipopolysaccharide exposure and in vivo in rodent models.

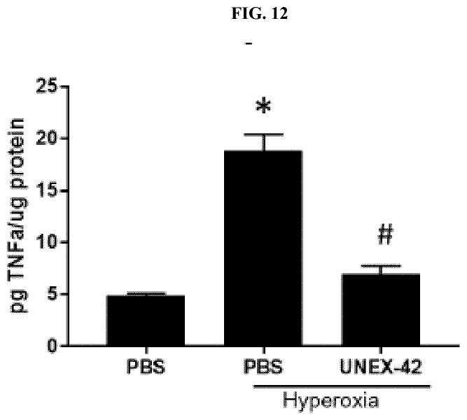

To assess the capacity of the herein disclosed EVs derived from MSC to prevent secretion of pro-inflammatory cytokines as a result of oxidative stress, A549 cells were primed with UNEX-42 for 3 hours, and then cultured in normoxia (21% O2) or hyperoxia (97% O2) for an additional 44 hours. The supernatant media was then collected and changes in secretion of the pro-inflammatory cytokines tumor necrosis factor alpha (TNFα), Exposure to hyperoxia increased secretion of each of these cytokines, while pretreatment with UNEX-42 attenuated these levels as shown in FIG. 12 and FIG. 13. In particular, the results depicted in FIG. 12 showed that UNEX-42 EVs suppress Hyperoxia-Induced Secretion of Tumor Necrosis Factor Alpha, and the results depicted in FIG. 13 showed that UNEX-42 EVs can suppress Secretion of Tumor Necrosis Factor Alpha induced by LPS.

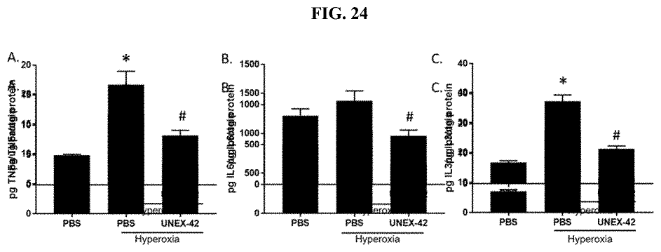

The effect of UNEX42 EVs on suppressing secretion of pro-inflammatory cytokines TNFa, IL6, and IL3 in human alveolar epithelial cells subjected to hyperoxia induced inflammation as shown in FIG. 24.

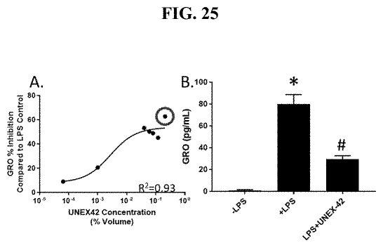

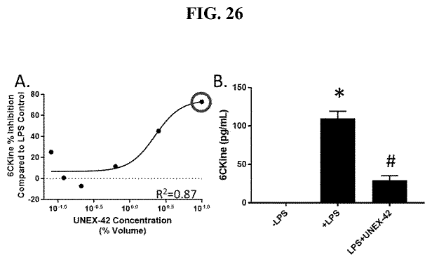

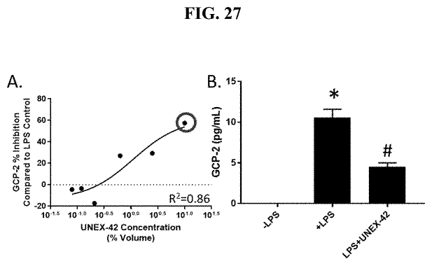

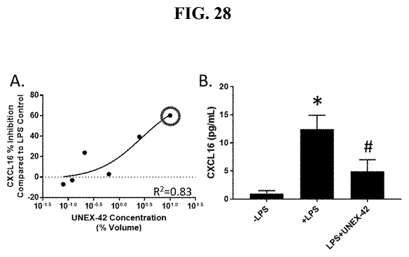

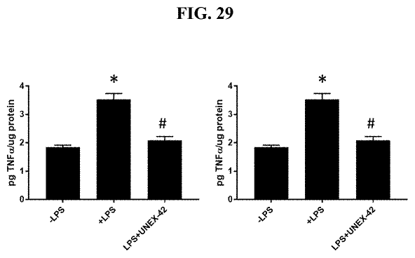

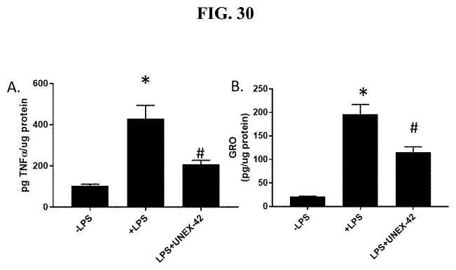

The effect of UNEX42 EVs on suppressing secretion of pro-inflammatory cytokines Chemokine (C-X-C Motif) Ligand 1 (GRO), Chemokine (C-C Motif) Ligand 21 (6CKine), Granulocyte Chemotactic Protein 2 (GCP2) Chemokine (C-X-C Motif) Ligand 16 (CXCL16) in human monocytes is shown in FIGS. 25-28. In addition, it was also found that UNEX-42 EVs inhibited LPS-Induced TNFa Secretion in Mouse Monocytes, and LPS-Induced TNFa and Chemokine (C-X-C Motif) Ligand 1 (GRO) secretion in Rat peripheral blood mononuclear cells (PBMCs) as shown in FIGS. 29 and 30, respectively.

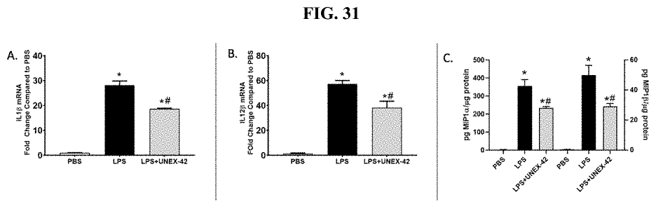

Exposure to LPS increased mRNA expression of interleukin 1 beta (IL1β) and interleukin 12 beta (IL12β) in human THP1 monocytes, while pretreatment with UNEX-42 attenuated mRNA expression of both as shown in FIGS. 31 (A) and (B), respectively. IL1β and IL12β are cytokine-encoding genes upregulated in animal models of ARDS, and these data support the potential for UNEX-42 to reduce inflammatory activation of circulating monocytes. Furthermore, UNEX-42 also attenuated secretion of the pro-inflammatory cytokines macrophage inflammatory protein 1 alpha (MIP1α) and beta (MIP1β) as shown in FIG. 31(C).

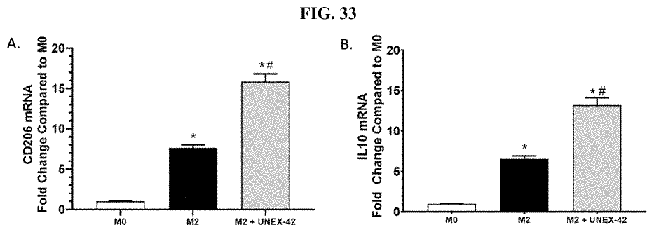

To study the effect of UNEX-42 EVs on expression of anti-inflammatory cytokines, THP-1 monocytes were polarized to MO macrophages, then to M2 macrophages via exposure to IL-4 and IL-13. Following M2 polarization, UNEX-42 was added, and gene expression was assessed. M2 polarization increased mRNA expression of mannose receptor (CD206) and interleukin 10 (IL10) anti-inflammatory cytokine, and the expression of both was further induced by UNEX-42 treatment as shown in FIG. 33.

These results demonstrated that the EVs derived from MSC have immunomodulatory activity by for example preventing secretion of pro-inflammatory cytokines in the lung in the setting of ventilation with supplemental oxygen.

To further assess the immunomodulatory capacity of the herein disclosed EVs derived from MSC, the effects of the EVs derived from MSC on activation of monocytes and macrophages were evaluated in vivo. In this experiment, human monocytes were pretreated with UNEX-42 for 3 hours prior to addition of LPS, a well-known activator of nuclear factor-kappa B (NF-κB)-mediated inflammatory cytokine production. UNEX-42 demonstrated attenuation of TNFα (FIG. 13). as shown in FIG. 13,

### Example 1.10 Studies of UNEX-42 Treatment in Animal Models

Rat Model of BPD

A rat model of BPD was developed for subsequent studies due to its larger size at birth compared to mouse, which allows for more reliable and consistent dosing and tissue evaluation. Sprague Dawley rat pups on PND 1 were housed in either normoxia or hyperoxia (92.5% O2). Nursing dams were rotated daily between litters in the normoxia and hyperoxia groups to avoid oxygen toxicity. Oxygen concentration was monitored using a representative in-cage real time monitor to confirm oxygen levels. PBS vehicle or UNEX-42 was administered via a single 50-uL IV injection, the route of administration to be used in the proposed clinical trial. Additionally, prior data has shown that a single IV dose of bone marrow MSC-derived conditioned media containing extracellular vesicles can result in improvements to lung architecture in a similar rat model of BPD.

A Rat Study Demonstrating a Reduction in Lung Inflammation

A study to assess UNEX-42 activity was designed to measure the infiltration of inflammatory cells into the lung. In this study, neonatal rat pups, along with a nursing mother, were randomly assigned to either normoxia or hyperoxia (92.5% O2) on PND 1 (See Table 6 below). On PND 2, PBS or UNEX-42 at a dose of 0.003×, 0.01×, 0.03×, 0.1×, 0.3× or 1× was administered as a single IV injection. Assessments on PND 8 included cell count and differential in bronchoalveolar lavage (BAL).

TABLE 5
Study design for assessing UNEX-42 activity based on measuring
the infiltration of inflammatory cells into the lung.

| | | | | Dose Total |
|---|---|---|---|---|
| Group | | | Dose | Phospholipid |
| Number | Oxygen | Treatment | Concentration | (pmol/kg) |
| G1 | Normoxia | PBS | NA | NA |
| G2 | Hyperoxia | PBS | NA | NA |
| G3 | Hyperoxia | UNEX-42 | 0.003X | 2.5 |
| G4 | Hyperoxia | UNEX-42 | 0.01X | 8.4 |
| G5 | Hyperoxia | UNEX-42 | 0.03X | 25.1 |
| G6 | Hyperoxia | UNEX-42 | 0.1X | 83.5 |
| G7 | Hyperoxia | UNEX-42 | 0.3X | 251 |
| G8 | Hyperoxia | UNEX-42 | 1X | 835 |

NA, not applicable;
PBS, phosphate buffered saline

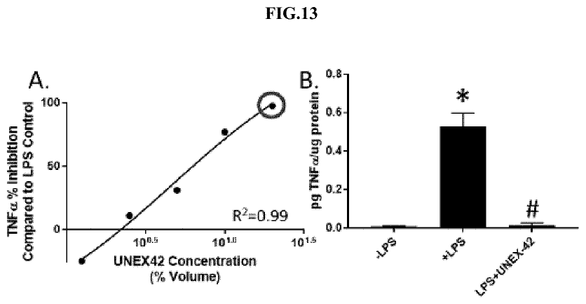

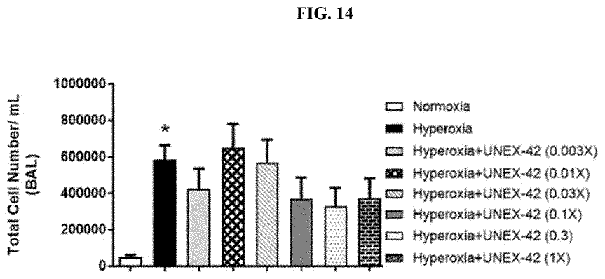

Exposure of neonatal rats to hyperoxia resulted in an approximately 11-fold increase in the total cell count in BAL as shown in FIG. 14, and administration of increasing doses of UNEX-42 reduced total cell count compared to hyperoxia controls. Neutrophil infiltration accounted for the largest differential, though these changes did not reach statistical significance (See Table 6 below).

TABLE 6
Lung infiltrating immune cells

| | | Cell Number/mL | | | |
|---|---|---|---|---|---|
| Treatment | | Total | | | |
| | UNEX-42 | Cell | | | |
| Oxygen | Dose | Number | Macrophage | Lymphocyte | Neutrophil |
| Normoxia | NA | 51667 | 51075 | 408 | 183 |
| Hyperoxia | NA | 585833 | 175700 | 3271 | 406863 |
| Hyperoxia | 0.003x | 425000 | 118275 | 2608 | 304117 |
| Hyperoxia | 0.01x | 664444 | 154889 | 5589 | 503967 |
| Hyperoxia | 0.03x | 570000 | 105713 | 4450 | 459838 |
| Hyperoxia | 0.1x | 367692 | 159031 | 2031 | 206631 |
| Hyperoxia | 0.3x | 328889 | 78178 | 1933 | 248778 |
| Hyperoxia | 1x | 391075 | 136950 | 2560 | 251565 |

NA, not applicable

This study demonstrates that hyperoxia increased infiltrating immune cells in the lung, and UNEX-42 generally decreased these numbers with increasing dose.

A Rat Study Demonstrating Improvements in Lung Architecture and Pulmonary Vasculature

A follow-up study of the pharmacodynamic effects of UNEX-42 on lung structure, as quantified by MLI, was conducted to further characterize the dose-response of UNEX-42, to generate an EC50, and to evaluate a wider range of doses (Study Report UNT-IFBPD-17). In this study, Neonatal rat pups, along with a nursing mother, were randomly assigned to either normoxia or hyperoxia (92.5% O2) on PND 1. On PND 2, PBS or UNEX-42 at a dose of 0.001×, 0.01×, 0.03×, 0.1×, 0.3×, or 1× (137 nM phospholipid) was administered as a single IV injection (See Table 7). Assessments occurred on PND 10 due to mortality concerns and included Fulton's index and lung architecture via histology and MLI, consistent with the terminal time point used in the study described above described in Table 6.

TABLE 7
Study design for the follow-up study of the pharmacodynamic
effects of UNEX-42 on lung structure.

| | | | | Dose Total |
|---|---|---|---|---|
| Group | | | Dose | Phospholipid |
| Number | Oxygen | Treatment | Concentration | (pmol/kg) |
| G1 | Normoxia | PBS | NA | NA |
| G2 | Hyperoxia | PBS | NA | NA |
| G3 | Hyperoxia | UNEX-42 | 0.001X | 0.92 |
| G4 | Hyperoxia | UNEX-42 | 0.01X | 9.2 |
| G5 | Hyperoxia | UNEX-42 | 0.03X | 27.7 |
| G6 | Hyperoxia | UNEX-42 | 0.1X | 92.2 |
| G7 | Hyperoxia | UNEX-42 | 0.3X | 277 |
| G8 | Hyperoxia | UNEX-42 | 1X | 922 |

NA, not applicable;
PBS, phosphate buffered saline

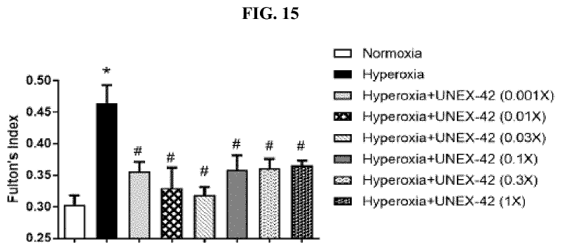

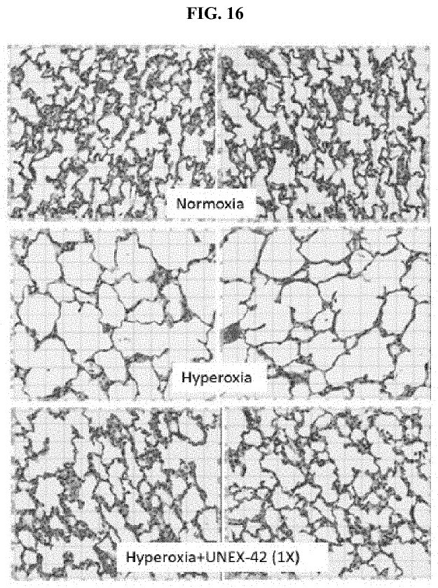

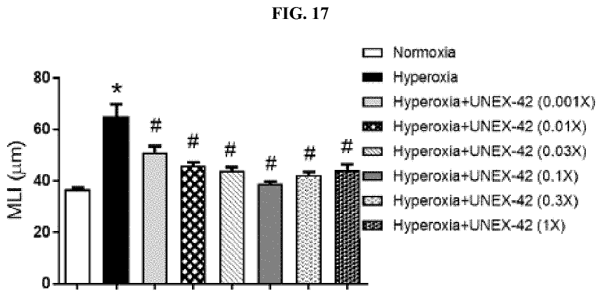

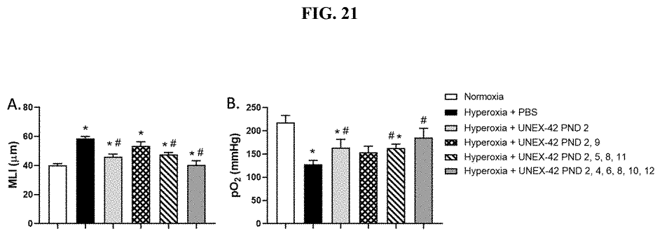

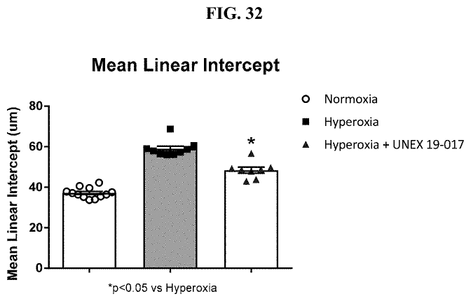

Animal survival in UNEX-42 treated groups was not statistically significantly different than the hyperoxia control group (Group 2). Exposure of neonatal rat pups to hyperoxia increased Fulton's index compared to normoxia control as shown in FIG. 15. UNEX-42 normalized Fulton's index in every dose tested, thus demonstrating maximal inhibition compared to hyperoxia control. With regard to lung architecture, hyperoxia exposure resulted in decreased lung alveolarization, characterized by fewer and larger alveoli as shown in FIG. 16. UNEX-42 treatment reversed this phenotype, as shown via improved histological appearance and improved MLI values at every UNEX-42 dose tested as shown in FIGS. 16, 17, and 32. Furthermore, as shown in FIG. 21, UNEX-42 multi-dose was most effective at reducing MLI (FIG. 21A) and increasing blood oxygen (FIG. 21B). The dosing regimen used for the experiment in FIG. 21 is shown in Table 8 below. Importantly, UNEX-42 improved changes in lung structure in a dose-responsive manner, with reductions of 22%, 29%, 32%, 40%, 35% and 32% compared to hyperoxia control at UNEX-42 doses of 0.001×, 0.01×, 0.03×, 0.1×, 0.3× and 1×, respectively.

TABLE 8
Study design for multi-dose UNEX-42 treatment

| Group | | | Dose | Number |
|---|---|---|---|---|
| Number | Model | Treatment | Concentration | of Doses |
| G1 | Normoxia | PBS | NA | 6 doses |
| | | | | (every other day) |
| G2 | Hyperoxia | PBS | NA | 6 doses |
| | | | | (every other day) |
| G3 | Hyperoxia | UNEX-42 | 137 nM | 1 dose |
| | | | phospholipid* | |
| G4 | Hyperoxia | UNEX-42 | 137 nM | 2 doses |
| | | | phospholipid | (every week) |
| G5 | Hyperoxia | UNEX-42 | 137 nM | 4 doses |
| | | | phospholipid | (every 3 days) |
| G6 | Hyperoxia | UNEX-42 | 137 nM | 6 doses |
| | | | phospholipid | (every other day) |

A Rat Study Demonstrating Improvements in Lung Function

Patients with BPD present with diminished lung function as measured by tidal volume and total lung capacity. Thus, potential functional benefits of UNEX-42 were evaluated using whole body plethysmography measurements, including measures of tidal volume, respiratory rate, and minute volume. Three doses of UNEX-42 were selected (0.01×, 0.1×, and 1×). These doses were selected based on the maximum effective dose from the previous study described above (0.1×), which was set as the middle dose. The high and low doses were set at 10-fold higher and lower, respectively.

Neonatal rat pups, along with a nursing mother, were randomly assigned to either continuous normoxia or hyperoxia (92.5% O2) on PND 1 (See Table 9 below). On PND 2, PBS or UNEX-42 at 0.01×, 0.1× or 1× (137 nM phospholipid) was administered as a single IV injection. Tidal volume, respiratory rate, and minute volume were assessed on PND 11, consistent with the target terminal timepoint in prior studies that examined lung architecture and vascular remodeling.

TABLE 9
Study design for evaluating potential functional benefits
of UNEX-42 by using whole body plethysmography measurements

| | | | | Dose Total |
|---|---|---|---|---|
| Group | | | Dose | Phospholipid |
| Number | Oxygen | Treatment | Concentration | (pmol/kg) |
| G1 | Normoxia | PBS | NA | NA |
| G2 | Hyperoxia | PBS | NA | NA |
| G3 | Hyperoxia | UNEX-42 | 0.01X | 8.4 |
| G4 | Hyperoxia | UNEX-42 | 0.1X | 84 |
| G5 | Hyperoxia | UNEX-42 | 1X | 840 |

NA, not applicable;
PBS, phosphate buffered saline

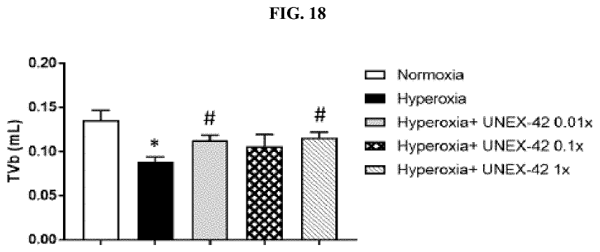

Exposure of neonatal rat pups to hyperoxia decreased tidal volume compared to normoxia control (FIG. 18), and UNEX-42 partially attenuated this at all doses, with 0.01× and 1× reaching statistical significance compared to hyperoxia control. These data support UNEX-42-mediated improvements in lung function following hyperoxia exposure.

A Rat Model of Pulmonary Arterial Hypertension

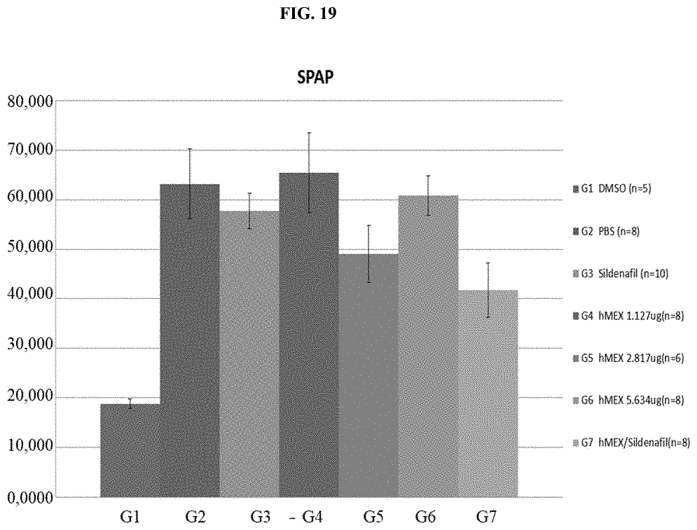

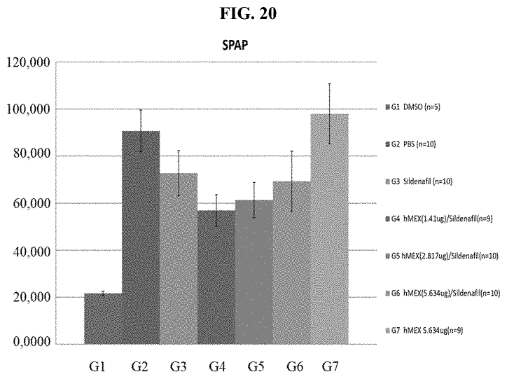

Exposure of rats to semaxinib/hypoxia (SU/hypoxia) resulted in increased systolic pulmonary arterial pressure (SPAP). The SU/hypoxia model rats were treated with UNEX-42 EVs alone or in combination with the phosphodiesterase type-5 (PDE5) inhibitor sildenafil. The benefit of UNEX-42 was evaluated by measuring the effect of the treatments on SPAP. As shown in FIGS. 19 and 20, the combination of UNEX-42 EVs and sildenafil reduced SPAP more than using sildenafil alone. The experimental setup for the results shown in FIG. 19 is shown in Table 10 below, and the experimental setup for the results shown in FIG. 20 is shown in Table 11 below.

TABLE 10
Treatment of SU/hypoxia model rats with
UNEX-42 EVs corresponding to FIG. 19.

| Group | | | Dose | | Number |
|---|---|---|---|---|---|
| Number | Model | Treatment | Concentration | | of Doses |
| G1 | DMSO | NA | NA | | NA |
| G2 | SU/Hypoxia | PBS | NA | | 9 doses |
| | | | | | (every 3 days) |
| G3 | SU/Hypoxia | Sildenafil | 30 | mg/kg | 54 |
| | | | | | (2X daily |
| | | | | | for 27 days) |
| G4 | SU/Hypoxia | UNEX-42 | 1.1 | μg | 9 doses |
| | | | | | (every 3 days) |
| G5 | SU/Hypoxia | UNEX-42 | 2.8 | μg | 9 doses |
| | | | | | (every 3 days) |
| G6 | SU/Hypoxia | UNEX-42 | 5.6 | μg | 9 doses |
| | | | | | (every 3 days) |

| | | | | |
|---|---|---|---|---|
| G7 | SU/Hypoxia | Sildenafil + | 30 mg/kg | 54 |
| | | UNEX-42 | Sildenafil; | Sildenafil; |
| | | | 5.6 μg | 9 doses |
| | | | UNEX-42 | UNEX-42 |

TABLE 11
Treatment of SU/hypoxia model rats with
UNEX-42 EVs corresponding to FIG. 20.

| Group | | | Dose | Number |
|---|---|---|---|---|
| Number | Model | Treatment | Concentration | of Doses |
| G1 | Normoxia | PBS | NA | 6 doses |
| | | | | (every other day) |
| G2 | Hyperoxia | PBS | NA | 6 doses |
| | | | | (every other day) |
| G3 | Hyperoxia | UNEX-42 | 137 nM | 1 dose |
| | | | phospholipid* | |
| G4 | Hyperoxia | UNEX-42 | 137 nM | 2 doses |
| | | | phospholipid | (every week) |
| G5 | Hyperoxia | UNEX-42 | 137 nM | 4 doses |
| | | | phospholipid | (every 3 days) |
| G6 | Hyperoxia | UNEX-42 | 137 nM | 6 doses |
| | | | phospholipid | (every other day) |

Study of Benefits of UNEX-42 EVs for Treatment of Idiopathic Pulmonary Fibrosis (IPF) in a Bleomycin Mice Model

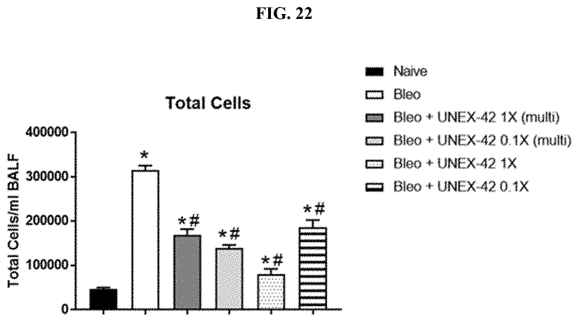

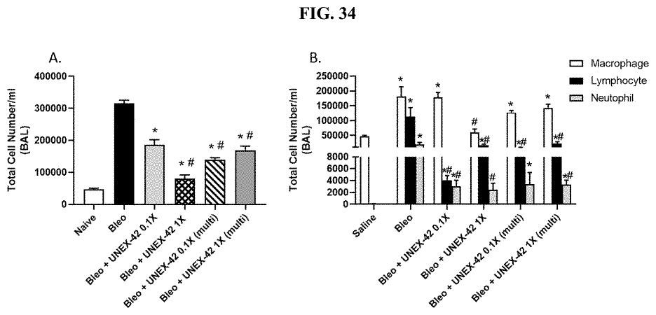

Bleomycin induced fibrosis was used as a model system for IPF. Table 12 below shows the study design for testing UNEX-42 EVs treating IPF in the bleomycin (bleo) model. UNEX-42 EVs reduced the number of immune cells infiltrating the bronchoalveolar lavage (BAL) in the bleomycin model for IPF as shown in FIG. 22 and FIG. 34. In particular, the results depicted in FIG. 22 shows the total number of cells in BAL, and demonstrated that UNEX-42 EVs reduced the total number in BAL in bleomycin treated mice. FIG. 34 depicts data evidencing that when compared with bleomycin control treated animals, administration of UNEX-42 EVs resulted in significant reductions in total cell counts in the BAL (FIG. 34A), which were predominantly due to reductions in neutrophils and lymphocytes, with lesser reductions in macrophages (FIG. 34B). It was also found that multi-dose UNEX-42 EV treatment resulted in minor improvements in alpha-SMA (alpha smooth muscle action) expression compared to single dose treatments.

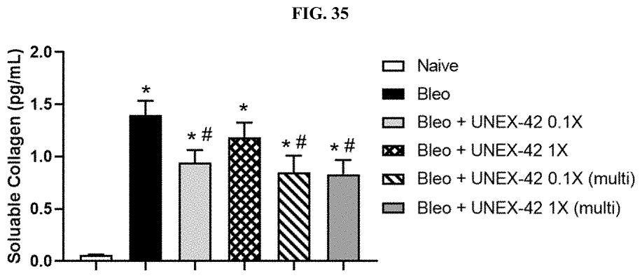

The advanced stage of acute respiratory distress syndrome (ARDS) is characterized by excessive deposition of extracellular matrix proteins, in particular collagen. Soluble collagen content was evaluated in the bronchoalveolar lavage fluid. Bleomycin administration increased BAL soluble collagen 23-fold compared to saline control (see FIG. 35). UNEX-42 reduced soluble collagen compared to disease control, with 0.1× single dose and 0.1× and 1× multi-dose groups reaching statistical significance (see FIG. 35). Single dose 0.1×UNEX-42 improved collagen content by 33%, 1× single dose by 15%, 0.1× multi-dose by 39 percent and 1× multi-dose by 40% compared to bleomycin control (see FIG. 35).

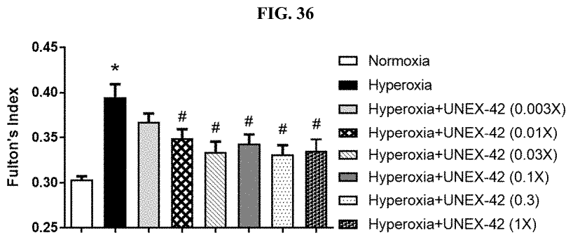

The impact of UNEX-42 on pulmonary vascular remodeling was evaluated after 8 days of hyperoxia exposure, and while hyperoxia increased Fulton's index above normoxia control, UNEX-42 partially reversed this effect at doses of 0.01× and higher (see FIG. 36). UNEX-42 improved Fulton's index by 7, 12, 16, 13, 16 and 15 percent at doses of 0.003×, 0.01×, 0.03×, 0.1×, 0.3× and 1× respectively as shown in FIG. 36.

TABLE 12
Study design for the bleomycin model of fibrosis

| Group | | | | Dose | Number |
|---|---|---|---|---|---|
| Number | Model | Treatment | | Concentration | of Doses |
| G1 | Saline | NA | | NA | NA |
| G2 | Bleomycin | PBS | | NA | 9 doses |
| | (1 U/kg) | | | | (every 4 days) |
| G3 | Bleomycin | 0.1X | UNEX-42 | 39.4 nM | 1 Dose |
| | (1 U/kg) | | | phospholipid* | |
| G4 | Bleomycin | 1X | UNEX-42 | 394 nM | 1 Dose |
| | (1 U/kg) | | | phospholipid | |
| G5 | Bleomycin | 0.1X | UNEX-42 | 39.4 nM | 9 doses |
| | (1 U/kg) | | | phospholipid | (every 4 days) |
| G6 | Bleomycin | 1X | UNEX-42 | 394 nM | 9 doses |
| | (1 U/kg) | | | phospholipid | (every 4 days) |

Silica Mice Model for Fibrosis

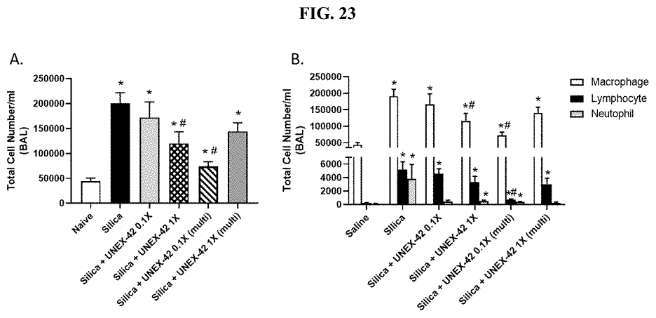

The benefit of UNEX-42 EVs for treating fibrosis was also shown in a silica model. The study design for the silica model experiments is shown in Table 13 below. Administration of silica resulted in a 4.5-fold increase in the total cell count in the BAL, and administration of UNEX-42 reduced total cell count when compared to administration of PBS, with 1× single-dose and 0.1× multi-dose groups reaching statistical significance (FIG. 23A). The 0.1× single-dose and 1× multi-dose groups demonstrated a reduction in cell count when compared with PBS treatment, but did not reach statistical significance. The largest change in BAL differential cell counts was observed in the macrophage and neutrophil counts (FIG. 23B). UNEX-42 EV treatment reduced total cell number in the BALF (bronchoalveolar lavage fluid) in the 1× single dose and 0.1× multi-dose groups as shown in FIG. 23.

It was also found that UNEX-42 EVs reduced the Ashcroft score of fibrosis in the 0.1× multi-dose group, and that UNEX-42 reduced a-SMA (alpha smooth muscle actin) staining in the 1× and 0.1× single dose groups.

TABLE 13
Study design for the silica model of fibrosis

| Group | | | | Dose | Number |
|---|---|---|---|---|---|
| Number | Model | Treatment | | Concentration | of Doses |
| G1 | Saline | NA | | NA | NA |
| G2 | Silica (25 | PBS | | NA | 4 doses |
| | mg/kg) | | | | (every 4 days) |
| G3 | Silica (25 | 0.1X | UNEX-42 | 39.4 nM | 1 dose |
| | mg/kg) | | | phospholipid* | |
| G4 | Silica (25 | 1X | UNEX-42 | 394 nM | 1 dose |
| | mg/kg) | | | phospholipid | |
| G5 | Silica (25 | 0.1X | UNEX-42 | 39.4 nM | 4 doses |
| | mg/kg) | | | phospholipid | (every 4 days) |
| G6 | Silica (25 | 1X | UNEX-42 | 394 nM | 4 doses |
| | mg/kg) | | | phospholipid | (every 4 days) |

A study of UNEX-42 EV toxicology was performed. The study design for evaluation toxicology of UNEX-42 EVs is shown in Table 14 below. There were no UNEX-42 related clinical signs, changes in body weights, hematology and clinical chemistry parameters, or organ weights. Similarly, there were no UNEX-42-related macroscopic or microscopic changes.

TABLE 14
Study design for toxicology evaluation of UNEX-42 EVs

| Group | | | Dose | Number |
|---|---|---|---|---|
| Number | Model | Treatment | Concentration | of Doses |
| G1 | NA | PBS | NA | 14 |
| | | | | (every other day) |
| G2 | NA | UNEX-42 | 137 nM | 14 |
| | | | phospholipid | (every other day) |

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains

All patents, patent applications, publications and references cited herein are incorporated by reference in their entirety to the extent as if they were individually incorporated by reference.

1. An isolated extracellular vesicle (EV), wherein the isolated EV contains one more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132.

2. The isolated EV of claim 1, wherein the EV contains one or more proteins selected from the group consisting of CD44, CD109, NT5E, MMP2 and HSPA8.

3. The isolated EV of claim 1, wherein the isolated EV is engineered to contain the one or more proteins.

4. The isolated EV of claim 1, wherein the isolated EV is obtained from a cell.

5. The isolated EV of claim 4, wherein the cell is selected from an immortalized cell line or a primary cell.

6. The isolated EV of claim 4, wherein the cell is a mesenchymal stem cell (MSC).

7. The isolated EV of claim 1, wherein the cell is a non-MSC.

8. The isolated EV of claim 7, wherein the non-MSC comprises a fibroblast cell, or a macrophage cell.

9. The isolated EV of claim 4, wherein the isolated EV contains an increased amount of the one or more protein markers compared to the average amount in all EVs obtained from the MSC.

10. The isolated EV of claim 9, wherein the isolated EV contains an at least 20% increased amount of the one or more protein markers.

11. The isolated EV of claim 6, wherein the MSC is isolated from Wharton's jelly, umbilical cord blood, placenta, peripheral blood, bone marrow, bronchoalveolar lavage (BAL), or adipose tissue.

12. The isolated EV of claim 1, wherein the isolated EV is a synthetic exosome produced in vitro.

13. The isolated EV of claim 12, wherein the synthetic exosome is a synthetic liposome.

14. The isolated EV of claim 1, wherein the isolated EV further contains Syntenin-1, Flotillin-1, CD105, and/or major histocompatibility complex class I.

15. The isolated EV of claim 1, wherein the isolated EV further contains a member of the tetraspanin family.

16. The isolated EV of claim 15, wherein the member of the tetraspanin family contains CD63, CD81, and CD9.

17. A method of isolating an extracellular vesicle (EV) having increased potency comprising engineering the EV to contain one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NTSE, HSPA8 (HEL-S-72p), RAB10, CD44, MMP2, CD109, and DKFZp686P132.

18.-33. (canceled)

34. A method of treating a disease or condition associated with reduced angiogenesis, acute inflammation, chronic inflammation, apoptosis, mitochondrial dysfunction, fibrosis or vasculopathy, comprising administering to a subject in need thereof isolated extracellular vesicles obtained from mesenchymal stromal cells, wherein the isolated extracellular vesicles comprise extracellular vesicles (EVs) containing one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132.

35.-112. (canceled)

113. A method of treating or preventing a respiratory disease or disorder, comprising administering to a subject in need thereof an effective dose of an isolated extracellular vesicle (EV), wherein the isolated EV contains one or more proteins selected from the group consisting of KRT19, TUBB, TUBB2A, TUBB2B, TUBB2C, TUBB3, TUBB4B, TUBB6, CFL1 (HEL-S-15), VIM, EEF1A1, EEF1A1P5, PTI-1, EEF1A1L14, EEFA2, ENPP1, NT5E, HSPA8 (HEL-S-72p), RAB10, CD44, MMP2, CD109, and DKFZp686P132.

114. The method of claim 113, wherein the respiratory disease or disorder comprises acute respiratory distress syndrome (ARDS), acute lung disease, asthma, chronic obstructive pulmonary disease, cystic fibrosis, pneumonitis, pulmonary fibrosis, acute lung injury, bronchitis, emphysema, bronchiolitis obliterans, or bronchopulmonary dysplasia (BPD).

115. The method of claim 113, wherein the method treats or prevents respiratory disease or disorder resulting from COVID-19.

116. The method of claim 114, wherein the pulmonary fibrosis is idiopathic pulmonary fibrosis.

117. The method of claim 113, wherein the respiratory disease or disorder is the result of an infection, sepsis, acid aspiration, or trauma.

118. The method of claim 113, wherein the infection is a bacterial infection or a viral infection.

119. The method of claim 113, wherein the respiratory disease or disorder is the result of a SARS-CoV-2 infection.

120. The method of claim 113, wherein the method comprises administering EV to a patient at risk of developing respiratory disease or disorder.

121. The method of claim 113, wherein the effective dose of the isolated EV is from 20 to 500 pmol of phospholipids of EVs per kg of subject being treated.

122.-131. (canceled)

132. The method of claim 113, wherein the isolated EV are administered parenterally.

133. The method of claim 113, wherein the method further comprises administering a phosphodiesterase type-5 (PDE5) inhibitor.

134. The method of claim 133, wherein the PDE5 inhibitor is sildenafil.

135. The method of claim 133, wherein the isolated EV and the phosphodiesterase type-5 (PDE5) inhibitor are administered in separate compositions, substantially simultaneously or sequentially.

136. The method of claim 133, wherein the isolated EV and the phosphodiesterase type-5 (PDE5) inhibitor are administered in the same composition.

137. The method of claim 133, wherein the isolated EV and PDE5 inhibitor are administered in one or more doses.

138. The method of claim 133, wherein the isolated EV and the PDE5 inhibitor are administered at an interval of 6 hours, 12, hours, 24 hours, 48 hours, 72 hours, 4 days, 5, days, 6 days, or once per week.

139. The method of claim 133, wherein the isolated EV is administered in 2 doses, 3 doses, 4, doses, 5 doses, 6 doses, 7 doses, 8 doses, 9 doses, 12 doses, 15 doses, or 18 doses 3 doses, 6 doses, 9 doses, 12 doses, 15 doses, or 18 doses, and wherein the PDE5 inhibitor is administered in 16 doses, 19 doses, 21 doses, 24 doses, 27 doses, 30 doses, 33 doses, 36 doses, 39 doses, 42 doses, 45 doses, 48 doses, 51 doses, 54 doses, 57 doses, 60 doses, 63 doses, or 66 doses.

140. The method of claim 113, wherein the isolated EV is administered once daily for 2 days, for 3 days, for 4 days, for 5 days for 6 days, or for a week.

141. The method of claim 113, wherein the method decreases systolic pulmonary arterial pressure (SPAP) in the subject.

142. The method of claim 113, wherein the method increases alveolar surface area of the lung in the subject.

143. The method of claim 113, wherein the method increases a concentration of blood oxygen in the subject.

144. The method of claim 113, wherein the method reduces inflammation in the lung in the subject.

145. The method of claim 113, wherein the method reduces deposition of extracellular matrix in the bronchoalveolar lavage fluid or in the lung.

146. The method of claim 113, wherein the method improves Fulton's index.

147. The method of claim 113, wherein the subject is a human, a non-human primate, a dog, a cat, a cow, a sheep, a horse, a rabbit, a mouse, or a rat.

148. The method of claim 133, wherein the PDE5 comprises sildenafil, vardenafil, zapravist, udenafil, dasantafil, avanafil, mirodenafil, or lodenafil.
